(19) United States
(12) Patent Application Publication
(10) Pub. No.: US 2025/0261870 A1
(43) Pub. Date: Aug. 21, 2025

(54) INTEGRATED PRESSURE AND FETAL HEART RATE MONITORING CERVICAL RIPENING CATHETER

(71) Applicant: Kamran Ghodsian, Newport Beach, CA (US)

(72) Inventors: Kamran Ghodsian, Newport Beach, CA (US); Steven Howard, La Jolla, CA (US); Keith Weier, San Marcos, CA (US)

(21) Appl. No.: 19/063,066

(22) Filed: Feb. 25, 2025

Related U.S. Application Data
(63) Continuation-in-part of application No. 17/510,915, filed on Oct. 26, 2021, now abandoned.
(63) Continuation-in-part of application No. 18/485,061, filed on Oct. 11, 2023.
Application No. 18/485,061 is a continuation of application No. 17/083,883, filed on Oct. 29, 2020, now Pat. No. 11,793,416.
Application No. 17/083,883 is a continuation of application No. 16/367,132, filed on Mar. 27, 2019, now abandoned.
(60) Provisional application No. 63/107,154, filed on Oct. 29, 2020.

(51) Int. Cl. A61B 5/03; A61B 1/303; A61B 5/00

(52) U.S. Cl. CPC A61B 5/035; A61B 1/303; A61B 5/6853; A61B 5/6875; A61B 2562/0247; A61B 2562/0271

(57) ABSTRACT

Cervical ripening catheters are disclosed, and methods for monitoring and measuring a change in intrauterine parameters without rupturing the amniotic sac. The catheter is provided with a uterine balloon, and a pressure sensor in pressure sensing communication with the uterine balloon. Sensing circuitry is configured to detect pressure applied to the pressure-sensor and communicate information relating to uterine contractions. The catheter may also be provided with sensors for determining fetal heart rate, oxygen saturation, respiratory rate, fetal blood pH or pelvimetry measurement. A working channel extends throughout the catheter to permit direct access to the amniotic sac by additional diagnostic or therapeutic tools.

### CROSS-REFERENCE TO RELATED APPLICATIONS

This application is a continuation-in-part of U.S. application Ser. No. 17/510,915, filed Oct. 26, 2021, which also claims benefit of U.S. Provisional Application No. 63/107,154, filed Oct. 29, 2020, the entirety of which is hereby incorporated by reference herein. This application is also a continuation-in-part of U.S. application Ser. No. 18/485,061, filed Oct. 11, 2023, which is a continuation of U.S. application Ser. No. 17/083,883, filed Oct. 29, 2020, now U.S. Pat. No. 11,793,416, issued on Oct. 24, 2023, which is a continuation of U.S. application Ser. No. 16/367,132, filed Mar. 27, 2019, the entirety of which is hereby incorporated by reference herein.

### BACKGROUND

### Technical Field

Embodiments of the present disclosure relate generally to processes of child-birth monitoring and assistance. More specifically, embodiments of the present disclosure relate to a system, apparatus, and method for monitoring and measuring a change in intrauterine pressure during labor, without rupturing the amniotic sac.

### Copyright And Trademark Notice

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of the embodiments disclosed herein to material associated only with such marks.

### DESCRIPTION OF THE RELATED ART

There are various reasons to induce labor. For example, when a woman is two or more weeks overdue (postdate), and labor does not start on its own, it may be desirous to induce labor, due to fetal or maternal indications, such as placental dysfunction, pregnancy induced hypertension, Preeclampsia, Diabetes, intrauterine growth restriction, conditions that may jeopardize fetal well-being, or other conditions that may affect the woman's health.

Labor may be induced by causing the cervix to soften and open. For example, the pharmaceutical substance, prostaglandin (PG), leads to local biochemical and biophysical alterations in the cervical region that reduce cervical resistance and induce myometrial contractions. Endocervical, or vaginal application of PG, in a gel form, is presently used for priming the cervix before labor induction and for labor induction. Currently, the Prepidil Gel is applied by intracervical injection using a syringe with a simple cannula. However, it is extremely difficult, if not impossible, to administer 3 ml of gel in a strictly endocervical fashion without applying some of the gel retroamniotically, which may cause side effects to uterine hypercontractility, and may lead to fetal distress.

Intracorporeal labor induction systems of the prior art do not control or measure uterus/amniotic-fluid pressure. By clogging the cervical canal, the existing systems make it hard, if not impossible, to insert a separate pressure measuring instrument into a patient's reproductive system to measure uterus and amniotic-fluid pressures. Furthermore, available uterus/amniotic-fluid pressure measurement devices are inherently inaccurate.

During many labor-inducing procedures, it is desirable to monitor and measure a condition within the uterus. However, known methods often rely on apparatus or devices that are inherently inaccurate and often cause or require the amniotic sac to be ruptured. An advantage of obtaining accurate measurements without breaching the amniotic sac is a decrease in the rate of intrauterine infection for the mother and decrease of infection for the fetus during labor. For example, it is desirable to evaluate the intrauterine pressure during labor before rupturing the amniotic sac. Current methods inadequately address this need.

Accordingly, the prior art inadequately addresses labor monitoring systems that: (i) may induce labor while accurately measuring the uterus and amniotic fluid pressure without rupturing the amniotic sac, (ii) facilitate control of the uterus and amniotic fluid pressure without rupturing the amniotic sac, and (iii) facilitate measuring and or observing a frequency of uterine contractions, without rupturing the amniotic sac.

Therefore, there is a need for a system, apparatus, and method that facilitates the monitoring and measuring of a condition inside the uterus during labor, without rupturing the amniotic sac. It is to these ends that some embodiments of the present disclosure have been developed.

### Summary of Some Example Embodiments

Disclosed herein are embodiments of systems, apparatus, and methods for child-bearing monitoring and assistance but more specifically, embodiments of a system, apparatus, and method for ripening a cervix and monitoring and measuring a condition inside the uterus without rupturing the amniotic sac. In exemplary embodiments an integrated pressure and fetal heart rate monitoring cervical ripening catheter includes sensors and structures adapted to determine pressure, fetal heart rate, SpO2, blood pressure, respiratory rate and pH of amniotic fluid. ECG sensors and ultrasound transducers may also be carried by the catheter, all wired back to the proximal end of the catheter which is provided with electrical connectors for removable electrical connection to one or more control units having a processor configured to process sensor data and display or otherwise output vital sign or other biometric data for an attending physician.

Methods include inserting the catheter through a cervix so that the balloon may be inflated and situated in the lower segment of the uterus, resting against the amniotic sac. Because the balloon of the catheter is in fluid communication with a pressure sensor, pulsations of the amniotic sac will be sensed by the sensing circuitry and displayed on a wired or wireless display screen. In this way, a change in intrauterine pressure during labor may be evaluated and monitored without rupturing the amniotic sac. The catheter includes a working channel opening distally through an exit port, for allowing direct access to the amniotic sac. A removable probe may be advanced distally through the working channel, the probe having a distal sensor such as a pH sensor, or ultrasound transducers for pelvimetry measurement.

Also disclosed herein are embodiments of a system for monitoring and measuring a change in intrauterine pressure during labor without rupturing the amniotic sac, may comprise: a catheter configured for manipulation by an operator, the catheter including: a distal end adapted to be placed at least partially inside a cervix of a patient; a uterine balloon situated substantially at the distal end of the catheter adapted to be placed at least partially inside a lower segment of a uterus of the patient; a first port situated at the proximate end of the catheter; a second port situated at the proximate end of the catheter; and a conduit fluidly communicating the first port and the second port to an interior of the uterine balloon, wherein the uterine balloon is inflated by introducing a fluid through the first port; and a pressure sensing module coupled to the second port, the pressure sensing module including an enclosure having a chamber in fluid communication with the uterine balloon via the conduit, a pressure-sensing membrane coupled to the chamber, and a sensing circuitry coupled to the pressure-sensing membrane configured to detect a pressure applied to the uterine balloon of the catheter.

Also disclosed herein are embodiments of a pressure sensing module for monitoring and measuring a change in intrauterine pressure during labor without rupturing the amniotic sac, may include: an enclosure configured to couple to a first port of a catheter, the catheter including: a distal end adapted to be placed at least partially inside a cervix of a patient; a uterine balloon situated substantially at the distal end of the catheter adapted to be placed at least partially inside a lower segment of a uterus of the patient; a second port situated at the proximate end of the catheter; and a conduit fluidly communicating the first port and the second port to an interior of the uterine balloon, wherein the uterine balloon is inflated by introducing a fluid through the first port; a chamber within the enclosure including a chamber port adapted for fluid communication with the uterine balloon via the conduit; a pressure-sensing membrane coupled to the chamber; and a sensing circuitry coupled to the pressure-sensing membrane configured to detect a pressure applied to the uterine balloon of the catheter.

Also disclosed herein are embodiments of a method for monitoring and measuring a change in intrauterine pressure during labor without rupturing the amniotic sac, may include the steps of: coupling a pressure sensing module to a catheter; inserting the catheter into a vagina and through a cervical canal of a female patient, wherein: a distal end of the catheter is placed inside the cervical canal, and a proximal end of the catheter is kept outside the cervical canal, the catheter including: a uterine balloon in fluid communication with the proximal end and attached at or near the distal end of the catheter, a first port situated at the proximate end of the catheter, a second port situated at the proximate end of the catheter, a conduit fluidly communicating the first port and the second port to an interior of the uterine balloon, and a vaginal balloon situated closer to the proximate end of the catheter than the uterine balloon and adapted to secure the uterine balloon inside the uterus; positioning the uterine balloon at a lower segment of a uterus of the female patient; inflating the uterine balloon from the proximal end and through the catheter by introducing a fluid through a first port of the catheter so that the fluid inflates the balloon and enters a first chamber of the sensing module; positioning the vaginal balloon at an external OS of the cervical canal of the female patient; inflating the vaginal balloon to anchor the uterine balloon inside the lower segment of the uterus so that the uterine balloon rests against an amniotic sac of the female patient; and reading a pressure output from the pressure sensing module.

In accordance with some embodiments of the present disclosure, there is provided an integrated pressure and fetal heart rate monitoring cervical ripening catheter. The catheter comprises an elongate, flexible tubular body, having a proximal end, a distal end and at least one working channel extending therethrough. At least a uterine balloon and preferably also a vaginal balloon are carried by the tubular body. An electronic component support such as a printed circuit board may be mounted on the distal end of the catheter, and having at least one emitter and at least one sensor. The working channel preferably extends distally through or around the electronic component support to preserve direct working channel access to the amniotic sac.

The component support may carry at least a first and second LED, and a photodetector, and the catheter and control unit may be configured to generate data convertible into useful vital sign or biometric information such as SpO2, fetal heart rate, or fetal blood pH. The support may optionally also carry at least one ultrasound transducer carried by the electronic component support.

The catheter may further comprise a maternal PPG sensor on the tubular body, proximal to the uterine balloon, configured for direct contact with maternal tissue. The catheter preferably further comprising a pressure sensor, for measuring pressure in the uterine balloon. The pressure sensor may comprise a pressure sensing balloon, which may be in pressure sensing communication with the uterine balloon. In one implementation, the pressure sensing balloon is located within the catheter shaft.

The pressure sensing balloon may extend between a proximal end and a distal end, and at least the distal end may be located distal to a proximal end of the uterine balloon. The pressure sensing balloon may be carried by a tubular shaft, and the pressure sensing balloon and tubular shaft may be positioned within an inflation lumen for the uterine balloon.

Various objects and advantages of the present disclosure will become apparent from the following description taken in conjunction with the accompanying drawings wherein are set forth, by way of illustration and example, certain embodiments of this disclosure. The drawings submitted herewith constitute a part of this specification, include exemplary embodiments of the present disclosure, and illustrate various objects and features thereof.

### BRIEF DESCRIPTION OF THE DRAWINGS

Elements in the figures have not necessarily been drawn to scale in order to enhance their clarity and improve understanding of the various embodiments disclosed herein. Furthermore, elements that are known to be common and well understood to those in the industry are not depicted in order to provide a clear view of the various embodiments disclosed herein.

FIG. 1 is a schematic diagram of the women's reproductive system during pregnancy.

FIG. 2 illustrates an embodiment of an inflatable system.

FIG. 3A illustrates an example of possible arrangements for fluid communication from a proximate end to a distal end of an embodiment of an inflatable system.

FIG. 3B illustrates an example of possible arrangements for fluid communication from a proximate end to a distal end of an embodiment of an inflatable system.

FIG. 3C illustrates an example of possible arrangements for fluid communication from a proximate end to a distal end of an embodiment of an inflatable system.

FIG. 3D illustrates an example of possible arrangements for fluid communication from a proximate end to a distal end of an embodiment of an inflatable system.

FIG. 4 illustrates an example embodiment of an inflatable system, shown inside a woman's reproductive system.

FIG. 5 illustrates an example embodiment of a system that employs a pressure sensing module coupled to a one-balloon catheter.

FIG. 6A illustrates an example embodiment of a system that employs a pressure sensing module coupled to a multi-balloon catheter.

FIG. 6B-6D illustrate an example embodiment of a stylet that may be implemented with some embodiments of a catheter disclosed herein, which includes a tubular support for facilitating the introduction of other instruments through the catheter.

FIG. 7 illustrates a block diagram of an example embodiment of a system employing a pressure sensing module.

FIG. 8A illustrates an example embodiment of a pressure sensing module.

FIG. 8B illustrates an example embodiment of a pressure sensing module.

FIG. 8C illustrates an example embodiment of a pressure sensing module.

FIG. 9 illustrates an example embodiment of a multi-balloon catheter system, shown inside a woman's reproductive system.

FIG. 9A illustrates an example embodiment of a method.

FIG. 10 illustrates an example embodiment of a method.

FIG. 11 is a perspective view of an embodiment of a multi functional catheter.

FIG. 12 is a side elevational view of a distal portion of the catheter shown in FIG. 11.

FIG. 13 is a cross-sectional view taken through the uterine balloon of FIG. 12.

FIG. 14 is a cross-sectional view taken through the catheter shaft of FIG. 11.

FIG. 15 is a longitudinal cross-section through a distal portion of the catheter of FIG. 11.

FIG. 16 is a distal end perspective view of an embodiment of a catheter.

FIGS. 17A and 17B are front perspective and rear perspective views of a distal electronic component subassembly in accordance with the present disclosure.

### DETAILED DESCRIPTION OF SOME EMBODIMENTS

In the following discussion that addresses a number of embodiments and applications of the concepts disclosed herein, reference is made to the accompanying drawings, which form a part thereof. Depictions are made, by way of illustration, of specific embodiments in which the concepts disclosed herein may be practiced; however, it is to be understood that other embodiments may be utilized, and changes may be made without departing from the scope of the present disclosure.

An integrated catheter is provided in the form of a multi-lumen device with intrauterine pressure, pulse oximetry and fetal heart rate monitoring capabilities. The catheter is introduced and used with the same protocol defined by Cook Medical, the manufacturer of a commercial cervical ripening catheter. Once the ripening catheter is inflated, the intrauterine pressure is monitored noninvasively prior to rupture of the amniotic membrane by monitoring the pressure waveform created inside the catheter to determine the frequency and intensity of contractions. The fetal heart rate is monitored noninvasively through the amniotic membrane with sensor(s) mounted to the distal end of the catheter. The sensor is encapsulated in the atraumatic distal tip of the catheter.

The sensor(s) are in electrical communication at the proximal end of the catheter to an electronics control module. The external control module houses the power supply and data processing and display capabilities for the sensor(s). All measurements may be recorded prior to the rupture of the amniotic membrane while providing mechanical ripening of the cervix. Additionally, a lumen in the catheter will be left open to enable endoscopic explorations throughout the procedure along with the use of a stylet for insertion, insertion and removal of a separate ultrasound probe or other diagnostic or therapeutic devices.

The heart rate sensor may be an optical based PPG (photoplethysmography) sensor that uses low power LED light sources of visible and/or infrared light and a photodetector to measure the volumetric variations of blood circulation. The application of this sensor against the amniotic membrane allows for direct monitoring of the fetal circulation without the complications of the maternal circulation.

Selected features of the catheter include:
Multi-lumen, sterile, single use silicone catheter.
Open working lumen for endoscopic, stylet and other device access.
Pressure monitoring sub-system with integrated liquid over air membrane.
Pressure-monitoring capability through connection to existing, reusable Koala cable connector in connection with existing perinatal monitors such as the GE Corometrics.
PPG sensor with multiple wavelength LED's at the distal end in close proximity to the amniotic membrane.
Fetal Heart Rate (FHR) monitoring through connection to a reusable electronics box that communicates to a computer.

Generally, the concepts disclosed herein involve various systems, apparatus, and methods for monitoring and measuring a condition inside the uterus without rupturing the amniotic sac, which in exemplary embodiments employs a pressure sensing module for use with a single or multi-balloon catheter. Coupled to the catheter, a pressure sensing module is configured with a plurality of chambers wherein at least a first of the plurality of chambers is adapted to receive a fluid that is applied via a catheter port and wherein at least a second of the plurality of chambers is adapted to contain air or another fluid separated from the first chamber by a membrane. A sensor coupled to the second chamber may be configured to detect a change in pressure applied to the membrane in order to communicate the pressure change to, for example, a pressure monitor of the system.

In some exemplary embodiments of the present disclosure, a system for inducing labor may include: a catheter with at least one, two or three balloons (or more) inflatable balloons that are configured to keep the system in an exact position within woman's reproductive system, monitor and/or control pressures inside uterus, exert controllable pressure on the cervical canal walls, stimulate cervical canal, monitor pressures inside cervical canal, and, if desired, administer pharmaceutical substances and/or rupture an amniotic sac.

In some exemplary embodiments of the present disclosure, a uterine balloon may be positioned at a proximal portion of the uterus, with respect to an operator, adjacent to the cervical internal orifice of the uterus (internal OS). In some embodiments, the uterine balloon may have different shapes so as to, for example, maximize the pressure against the decidua and the external orifice of the uterus (external OS) and to minimize the pressure on the fetal head. In other embodiments, the uterine balloon may have different surface roughness, for example, for anchoring the uterine balloon in place.

In some exemplary embodiments of the present disclosure, the system may optionally or additionally include a vaginal balloon for positioning in the vagina for applying pressure on the external OS. The combination of a uterine balloon and a vaginal balloon may stabilize the position of the inflatable system within the woman's reproductive system as long as both balloons are inflated.

In some exemplary embodiments of the present disclosure, the system may optionally or additionally include a cervical balloon for positioning in the cervical canal, shaped so as to maximize the contact area with the cervix. The shape and the surface roughness of the cervical balloon may also be designed in order to maximize cervix contact and stimulation.

In exemplary embodiments, the one or more balloons of the system may stimulate the secretion of hormone by exerting pressure on the proximal decidual surfaces of the uterus and on the cervix, to soften and ripen the cervix, to cause the cervix to dilate, and to induce labor. The balloons, which in some embodiments may have rough external surfaces in order to keep them anchored in place, may be inflated by the operator directly after their insertion, or manually and gradually by the patient.

In some exemplary embodiments of the present disclosure, various sensors and instruments may be used as part of the system to monitor cervical dilation, fetal well-being, and the woman's conditions. In some embodiments, pharmaceutical substances may also be applied to the cervix canal through a cervical canal portion of the disclosed device.

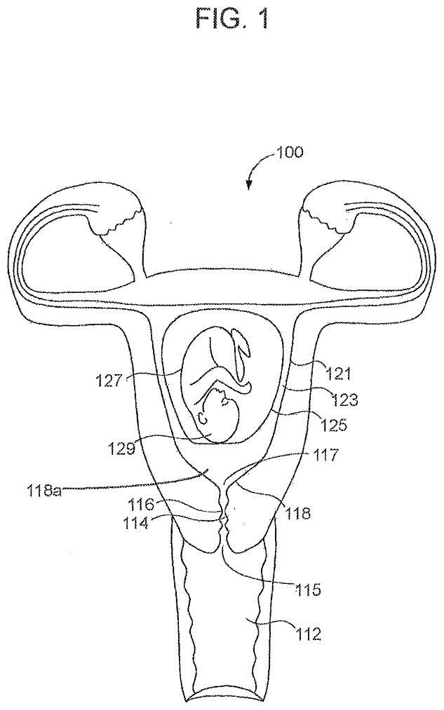

Turning now to the figures, FIG. 1 is a schematic diagram of the women's reproductive system during pregnancy. More specifically, FIG. 1 illustrates a woman's reproductive system 100 during pregnancy, showing a vagina 112, a cervix 116 forming a cervical canal 114, an ectocervix or external OS 115, an internal OS 117, a uterus 118, an endometrium 121 which is the mucous membrane lining the uterus 118, an amniotic sac 125 containing a fetus 127 having a head 129, and a decidua 123 which is the mucous membrane lining the uterus 118 in preparation for, and during pregnancy.

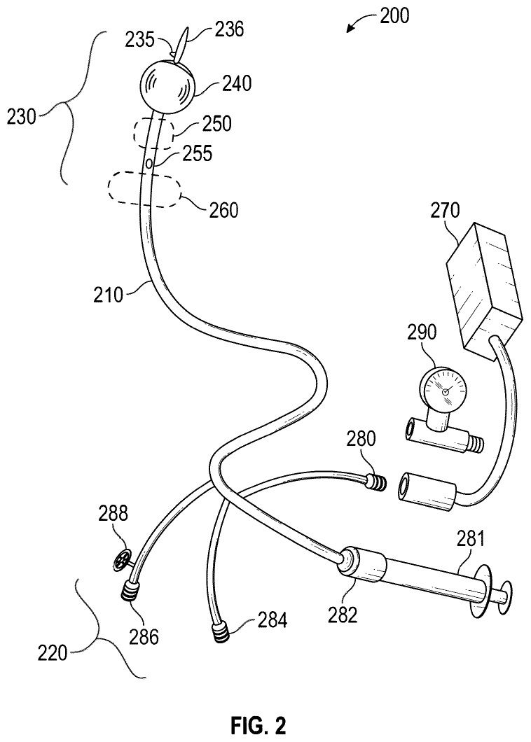

Turning now to the next figures, FIG. 2 illustrates an inflatable system in accordance with the present disclosure. More specifically, FIG. 2 schematically illustrates an embodiment of system 200 for cervical dilation, labor induction and uterus and cervical canal pressure monitoring and control. As seen in FIG. 2, system 200 includes a catheter 210 having a proximal end 220 and a distal end 230, with respect to an operator, wherein the distal end 230 enters a woman's reproductive system and the proximal end 220 remains outside her body to be manipulated by an operator/medical staff.

In some exemplary embodiments, catheter 210 may include: (1) a uterine balloon 240 substantially at the distal end 230 and a conduit/tube (not shown) inside the catheter 210, spanning from the proximal end 220 to the distal end 230 and in fluid communication with the uterine balloon 240. In some exemplary embodiments, the catheter 210 may also include a cervical balloon 250, in fluid communication with a cervical-balloon conduit/tube (not shown) inside the catheter 210, spanning from the proximal end 220 to the distal end 230.

As seen in FIG. 2, the location of the cervical balloon 250 on the catheter 210 is closer to catheter's proximal end 220 than the location of the uterine balloon 240 is. The catheter 210 is designed for insertion into the woman's reproductive system, so that the cervical balloon 250 is positioned and inflated within the cervical canal 114. The cervical balloon 250 is designed for applying pressure on the cervix 116. It will be appreciated that the pressure on the cervix 116 may further include pressure on the internal OS 117 and on the external OS 115.

In some embodiments, the inflatable system 200 may include a vaginal balloon 260, in fluid communication with a vaginal-balloon conduit/tube (not shown) inside the catheter 210, spanning from the proximal end 220 to the distal end 230. The vaginal balloon 260 is adapted for positioning within the vagina 112, at a distal portion thereof, for pressing against the external cervical OS 115. The one, two or three-balloons of system 200, from catheter 210's distal end 230, may be inserted into a woman's reproductive system 100, prior to inflation of the balloons.

In an exemplary three-balloon inflatable system 200, the uterine balloon 240 and the vaginal balloon 260 are partly operative to anchor the cervical balloon 250 in place and to enhance hormonal secretion by applying pressure on the decidual 123, the internal OS 117, and the external OS 115. At the same time, the cervical balloon 250, anchored within the cervical canal 114, may accelerate cervical dilation, by applying pressure on the cervix 116, and preferably also, the internal OS 117 and on the external OS 115.

In various embodiments the inflatable system 200 may also have openings, such as opening 255, which is in fluid communication with a conduit/tube (not shown) inside the catheter 210, spanning from the proximal end 220 to the distal end 230. Opening 255 is situated on the catheter 210 such that, for example, upon placement in a woman's reproductive system the opening 255 is positioned within the cervical canal 114 and is used to insert pharmaceutical substances into the cervical canal 114.

In accordance with the present disclosure, the inflation of the balloons of the inflatable system 200 may be performed by a doctor or a midwife (not shown) directly after its insertion. In some exemplary embodiments, the inflation of the balloons of the inflatable system 200 may be performed by the patient via a hand pump 270, in a gradual manner, for example over several hours. Alternatively still, uterine balloon 240 and or vagina balloon 260 may be inflated by the doctor or midwife, while the inflation of the cervical balloon 250 may be performed by the patient, for example, via a hand pump 270. Without limiting the scope of the present disclosure, pump 270 in FIG. 2 represents any kind of manual or automatic gas or fluid pump suitable for inflating the one or more balloons of catheter 210 of system 200.

Having a pressure sensor at the distal end of the inflatable instrument, inside the uterus and/or cervix canal, has some limitations and disadvantages including: (1) the pressure sensor must be small enough, such as a few millimeters, to fit and/or move inside the instrument, which has to pass through the cervix canal; (2) the pressure sensor must be disposable since the entire intracorporeal part of the system needs to be discarded after use; (3) the sensed pressures need to be transmitted outside the woman's body for recording or display, by wire or wirelessly; and (4) the system will be more expensive and bulkier as a result of the mentioned limitations and disadvantages.

Accordingly, a system for labor induction and monitoring/control in accordance with the present disclosure will be much smaller, less expensive and simpler if the pressures inside the uterus and/or cervical canal can be transferred to one or more sensors situated outside the woman's body. To do so, a very simple and instantaneous transfer of pressure is possible by using gasses and fluids. However, pressure transfer through a gaseous medium is not as accurate as through incompressible fluids since gasses are compressible and their compressibility varies by temperature and pressure, which introduce undesirable variables into the sensing subsystem. On the other hand, pressure may be accurately sensed at any point of a fluid body without any erroneous effect from the surrounding variables. Therefore, the preferred embodiment of this specification inflates, at least, the uterine balloon by nontoxic incompressible fluids such as water.

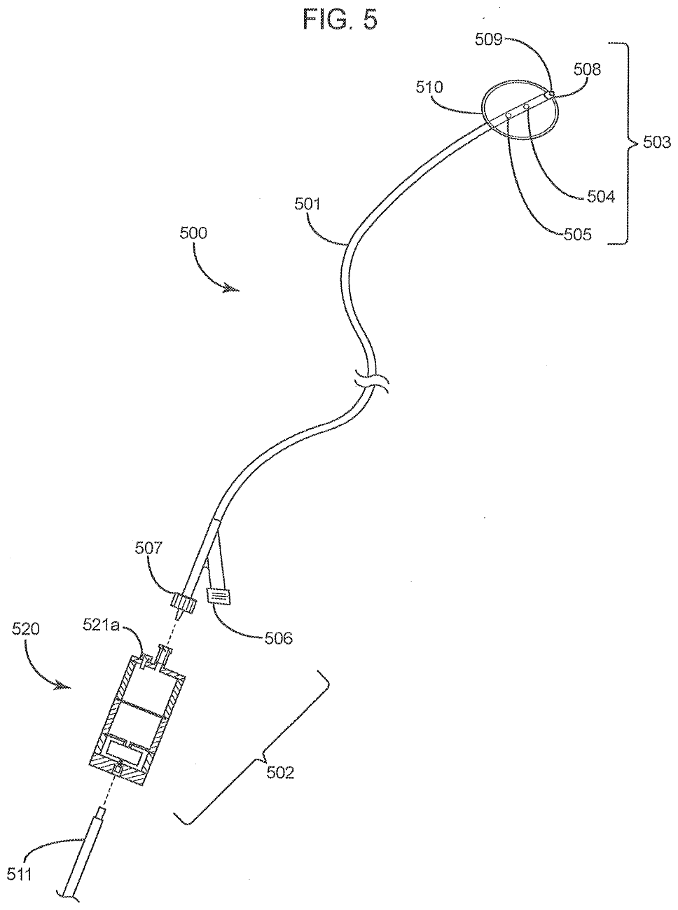

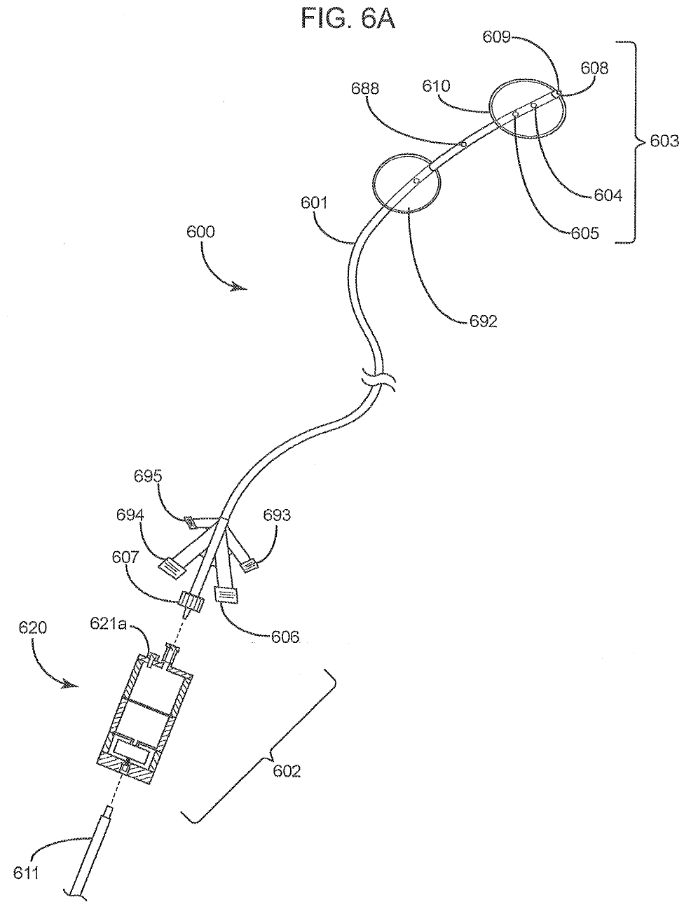

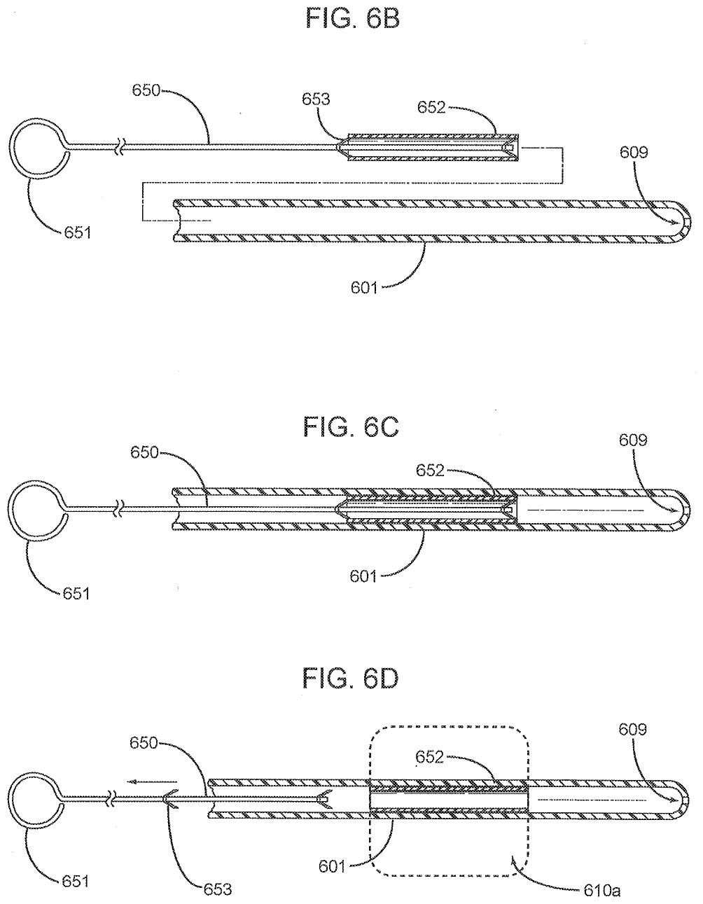

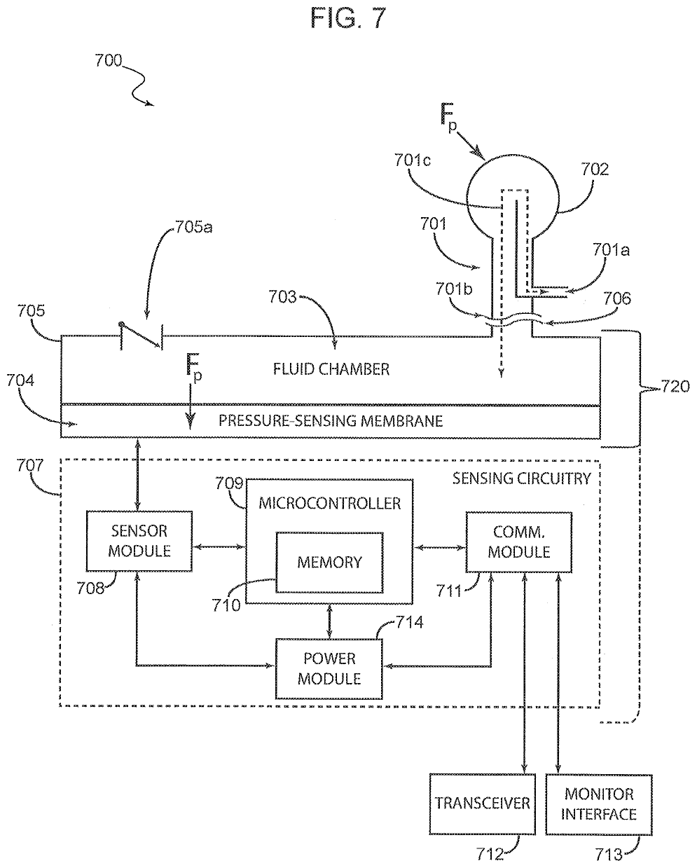

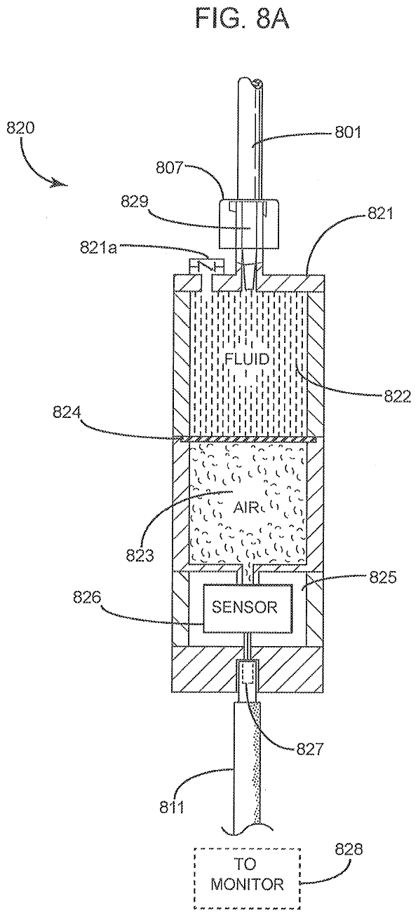

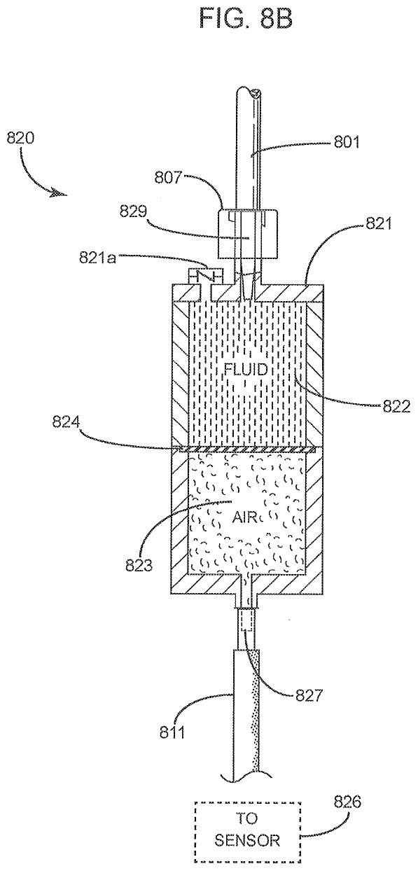

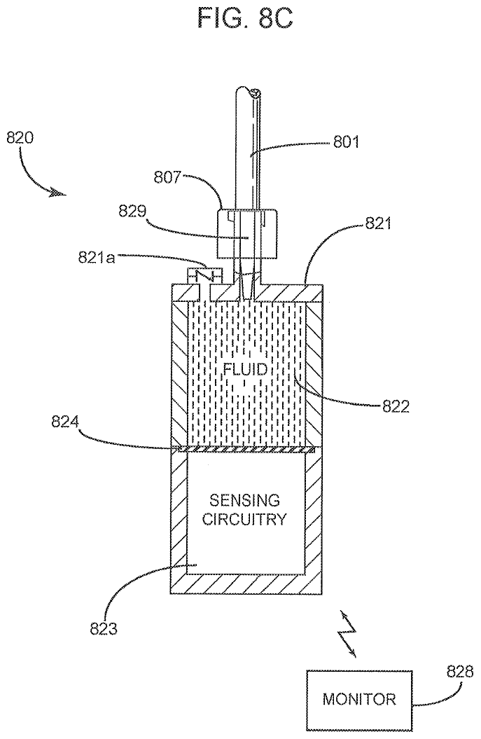

In the uterus, as a result of hydraulic principles, the pressure inside the fluid-filled uterine balloon will rapidly come to static equilibrium with the pressures of the amniotic sac and the uterus walls. At such stage, the pressure of the amniotic fluid, the uterine balloon fluid and the pressure on the uterus walls are equal. Hence a sensor outside the woman's body and in fluid contact with the uterine balloon will sense the exact pressure of the uterus/amniotic fluid. The uterus/amniotic fluid pressure may be controllably increased or decreased by inflating or deflating the uterine balloon, respectively. While inflating or deflating the uterine balloon, the exact pressure inside the uterus may also be sensed and observed. With fluid, no wait time is required for pressure equilibrium. A pressure sensing module that may be used with system 200 or other systems in accordance with exemplary embodiments of the present disclosure, is discussed further below with reference to FIG. 5, FIG. 6A, FIG. 7, and FIGS. 8A-8C.

The same is true about a cervical balloon and the monitoring and control of the cervix pressures. In some embodiments, the fluid inside the cervical canal balloon may even be pulsated to further stimulate the canal walls. The pulsation may be caused manually or mechanically. Here also, the cervical balloon itself may be a combination of multiple balloons which may be inflated separately or as a group.

The inflation/deflation of multiple balloons may be controlled together or independently to allow stimulation of the cervical canal with variable pressures across both time and position within the cervical canal. Such an arrangement may be used to create a pressure signal, varying over time and space, to stimulate different regions within the canal at different pressures and at different times, which may be helpful in stimulating dilation more effectively.

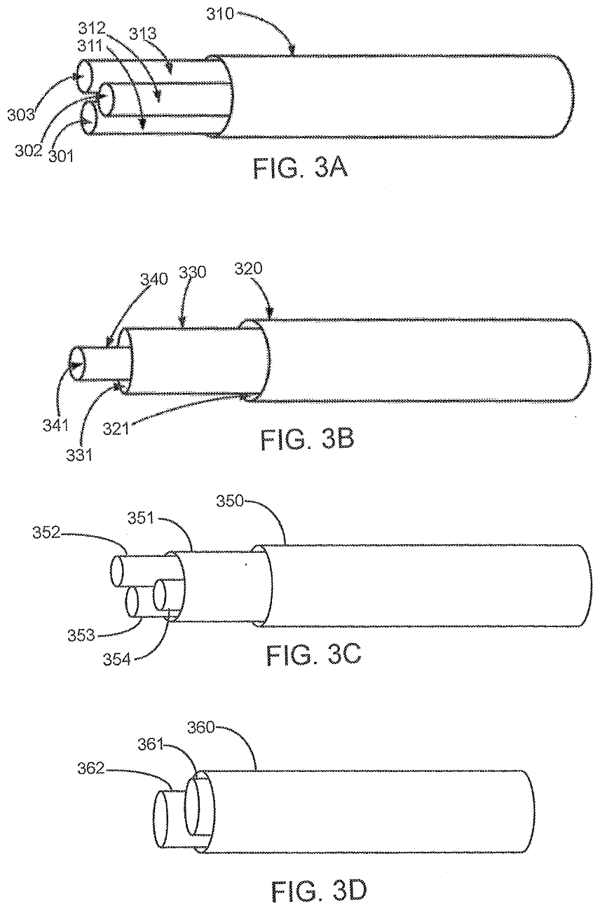

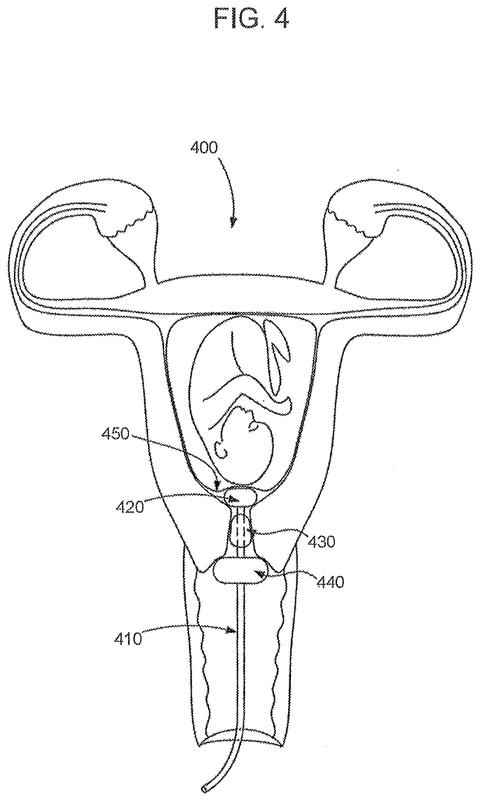

For example, if two balloons are employed, their inflation and deflation may be alternated to create a two-point variable pressure signal. In some embodiments, the inflatable system 200 may be coupled with a pressure controller to automatically inflate and deflated the multiple balloons according to a predefined signal pattern chosen by the operator. For example, the pressure controller may have several settings such as sinusoidal pattern, ramped inflation/deflation, and pulsed/rapid inflation/deflation, each of which may be selected by the operator to inflate and deflate the balloons accordingly for different types of stimulation. In multiple balloon embodiments, each balloon may need its own separate micro-tube for independent inflation/deflation, in which the micro-tubes pass through a larger outer tube to allow easy handling and control of the inflatable system, in a manner similar to those shown in FIGS. 3A-3D discussed below.

In general, a vaginal balloon in accordance with the present disclosure need not be inflated by fluid because it is usually not used to sense or control any pressure; however, if fluid is used to inflate the other balloons, it may be easier to do the same with the vaginal balloon. The vaginal balloon may also consist of more than one balloon controlled as a group or separately in a manner similar to the cervical canal balloons described above.

In exemplary embodiments, at least one of the balloons is inflated by fluid such as water to accurately measure and control the pressure of its surrounding. An increase or decrease of the size of the balloons will increase or decrease, respectively, the pressure of their environment which at the same time can be measured. For example, a small fluid inflation of the uterine balloon 240 enables an accurate measurement of the uterus and amniotic fluid pressures while any increase in the uterine balloon size will increase the uterus and amniotic fluid pressures. Same is true with cervical canal pressure measurement and control. Usually there is no need for measurement and/or control of vaginal pressure and therefore the vaginal balloon may be inflated by air instead of a fluid.

FIG. 2, shows an example system 200 with four connection ports 280, 282, 284, and 286, to which gas and fluid pumps, sensors and gauges, and other instruments may be attached. Each port may be of different kind and use different operating mechanisms. For example, connection port 280 may be closed in its unattached state and be open as long as being connected to any device. Or, for example, port 286 may have a manually operated valve 288.

Therefore, connection port 280, which in this example is assumed to be in fluid communication with uterine balloon 240, may first be connected to a pressure gauge 290 and a fluid pump 270 until the uterine balloon 240 is inflated to a desired pressure or size and subsequently be disconnected from the pump, if needed. In such a case, the uterus pressures can be continuously monitored. If constant monitoring of the pressure is not needed, both the pressure gauge 290 and the fluid pump 270 may be removed and the pressure of uterus be checked at the connection port 280 whenever desired. In some embodiments, the conduit/tube connecting connection port 280 to uterine balloon 240 may even have a separate dedicated port for connecting to sensors and gauges. In yet another example, connection port 280 may be attached to an automated machine for monitoring and/or controlling the pressure of the uterus. In various embodiments, dedicated monitoring ports for each balloon may be connected to a monitoring device(s) while ports, such as port 280, are dedicated to injection, manipulation and/or pumping devices. In these embodiments the pumping device can be a mere syringe 281. There is no need for complicated pumping devices with pressure gauge. Assuming that connection port 284 is in fluid communication with cervical balloon 250, it can be connected to a fluid pulsating device that is capable of controlled fluid pulsation with desired low and high pressures.

If fluid is used to inflate the balloons, there are several ways to eliminate the air in the conduits/tubes. In one embodiment the conduits and the balloons of the system 200 may be manufactured in a collapsed or vacuumed initial form. In other embodiments the connection ports may provide manual release valves to empty the trapped air while inserting fluid into the conduits. Such solutions are known to those skilled in the art and need no further elaboration.

As illustrated in FIG. 2, different devices may be used as a part of such inflatable system 200. In various embodiments the inflatable system 200 may include at least one additional conduit/tube (not shown), having at least one opening 235, to the uterus. The at least one additional conduit/tube is operable for inserting at least one device 236 to the woman's reproductive system. In the embodiment of FIG. 2, the at least one additional tube is operable for inserting the at least one device 236 into the uterus, via the opening 235. Similarly, another conduit/tube may be used for inserting another device into the cervical canal, via a similar opening. In FIG. 2, the at least one device 236 is a device for rupturing the amniotic sac. Device 236 may be operated as a plunger-like device, using a handle at the proximal end 220 of the inflatable system 200. After the puncture, device 236 may be withdrawn from one of the connection ports.

In yet other embodiments, the at least one device 236 may be a sensor, for example, for sensing amniotic-fluid temperature, or for sensing fetal heart-beat, preferably operable via the opening 235 to the uterus. The measurements may be transmitted extracorporeally in a wired or wireless manner.

It will be appreciated that the device 236 may be selected from the group consisting of device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, a device for scalp blood pH sampling, and the like.

Alternatively, the at least one device 236 may include a drug form, designed for passive dispensing of a medication. The drug form may be inserted with a syringe 281, and the syringe 281 may then be withdrawn. The passive dispensing of a medication may be by instantaneous release, delayed release, pulsating release, timed release, slow release, or another release form, as known, operable via the opening 235 to the uterus, or via a similar opening(s) to the cervical canal. Device 236 may be electronically-controlled, pre-programmed, or may be wirelessly controlled from an extracorporeal station.

In yet other embodiments, device 236 may include a device for measuring an extent of cervical dilation, operable via an opening to the cervical canal. The measurements may be transmitted extracorporeally by wire or wirelessly. In various embodiments, device 236 may be a combination of several devises and/or sensors.

FIG. 3A-FIG. 3D illustrate several exemplary embodiments of conduits for a catheter in accordance with the present disclosure. In FIG. 3A, a first tube 310 may include separate conduits 301, 302, and 303 corresponding to separate tubes 311, 312, and 313, all of which reside within a conduit of tube 310. In exemplary embodiments, each of tubes 311, 312, and 313 may connect to a connection port, to a balloon or an opening via their own respective conduits 301, 302, and 303.

As will be evident from the following figures, other configurations of tubes and conduits may be possible without deviating or limiting the scope of the present disclosure. In FIG. 3B, tubes 340 and 330 may be nested within tube 320, each of the tubes providing concentric conduits 341, 331, and 321, respectively. In other embodiments the arrangement of the tubes may be a combination of the arrangements in FIGS. 3A and 3B. For example, and without limiting the scope of the present disclosure, FIG. 3C shows tube 350 housing tube 351, which in turn houses several tubes 352, 353, and 354. FIG. 3D shows yet another embodiment, in which a single catheter tube may include two smaller tubes with conduits therein, such as tube 360 housing tube 361 and tube 362.

Turning now to the next figure, FIG. 4 depicts a three-balloon catheter 410 that has been inserted into a woman's reproductive system 400 and subsequently inflated, so that the uterine balloon 420 is in contact with the amniotic sac 450 in the lower segment of the uterus, the cervical balloon 430 is in the cervical canal, and the vaginal balloon 440 is in the distal end of the vagina.

In the example of FIG. 4, the uterine balloon 420 applies pressure on the decidua and the internal OS. The cervical balloon 430 applies pressure on the cervix walls, and the vaginal balloon 440 applies pressure on the external OS. For withdrawal, the balloons are deflated.

As seen from FIG. 4, the uterine balloon 420 is operative partly to separate the amniotic sac 450 from the decidua in the lower segment of the uterus, thereby stimulating endogenous hormone secretion of PG from the decidua. The hormone secretion by the decidua is operative to soften and ripen the cervical canal and induce labor. Similarly, the cervical balloon 430 is operative to stimulate hormone secretion by the cervix. Again, the hormone secretion is operative to soften and ripen the cervical canal and induce labor.

In some embodiments electrocardiogram of the fetus heart may be obtained by temporarily or permanently attaching a desired type and number of electrodes on the surface of the uterine balloon 420, to be directly in contact with the fetus head or indirectly through the amniotic sac, and to send the sensed signals to an extracorporeal EKG or ECG machine, by wire or wirelessly. In some embodiments the electrodes may be a part of or be ingrained in the material of the uterine balloon 420.

Turning now to the next figure, FIG. 5 depicts system 500, which includes a catheter 501 having a proximal end 502 and a distal end 503, with respect to an operator, wherein the distal end 503 enters a woman's reproductive system and the proximal end 502 remains outside her body to be manipulated by the operator, such as a physician or medical staff.

In some exemplary embodiments, catheter 501 may include: a single balloon 510 that serves as a uterine balloon, substantially at the distal end 503 of catheter 501; a conduit (not shown) inside the catheter 510, spanning from the proximal end 502 to the distal end 503 in fluid communication with balloon 510 via said conduit with openings 504 and 505; a first port 506 for introducing a fluid into the balloon 510; and a second port 507 coupled directly to a pressure sensing module 520. Catheter 501 may include a tip 508 that is blunt so as to not rupture the amniotic sac or may be alternatively configured to rupture the amniotic sac. The blunt tip 508 may include an opening 509 so that an instrument, such as a temperature transducer or an imaging transducer, may be provided access to the lower segment of the uterus and amniotic sac of a patient via opening 509. Moreover, as will be explained below, in some exemplary embodiments, pressure sensing module 520 may be coupled to an external component such as a sensing device or monitor via communication means 511.

In exemplary embodiments, system 500 includes a catheter 501 configured for manipulation by an operator, the catheter including: a distal end 503 adapted to be placed at least partially inside a cervix of a patient; a balloon 510 situated substantially at the distal end 503 of the catheter 501 adapted to be placed at least partially inside a uterus of the patient; a first port 506 situated at the proximate end of the catheter 501; a second port 507 situated at the proximate end 502 of the catheter 501; and a conduit fluidly communicating the first port 506 and the second port 507 to an interior of the balloon 510, wherein the balloon is inflated by introducing a fluid through the first port 506; and a pressure sensing module 520 coupled to the second port, the pressure sensing module 520 including an enclosure having a first chamber in fluid communication with the conduit, a second chamber in fluid communication with a sensor, and an pressure-sensing membrane separating the first chamber from the second chamber, wherein the pressure sensing module is configured to detect a pressure applied to the balloon of the catheter.

In exemplary embodiments of the present disclosure, the conduit of the catheter 501 includes: a first conduit communicating the first port 506 to a first opening 504 on the distal end of the catheter situated at the interior of the balloon 510; and a second conduit communicating the second port 507 to a second opening 505 on the distal end of the catheter situated at the interior of the balloon 510.

In exemplary embodiments of the present disclosure, the first port 506 of the catheter may include a valve configured to prevent a fluid injected through port 506 and into the conduit that fills the balloon 510 from returning back out of port 506. Similarly, a first chamber of pressure sensing module 520 may also include a valve, such as a check-valve or otherwise scalable valve 521a, which allows any air inside the chamber to exit the pressure sensing module as the fluid is received. Once adequately filled, a user may seal the valve 512a in order to maintain the fluid within.

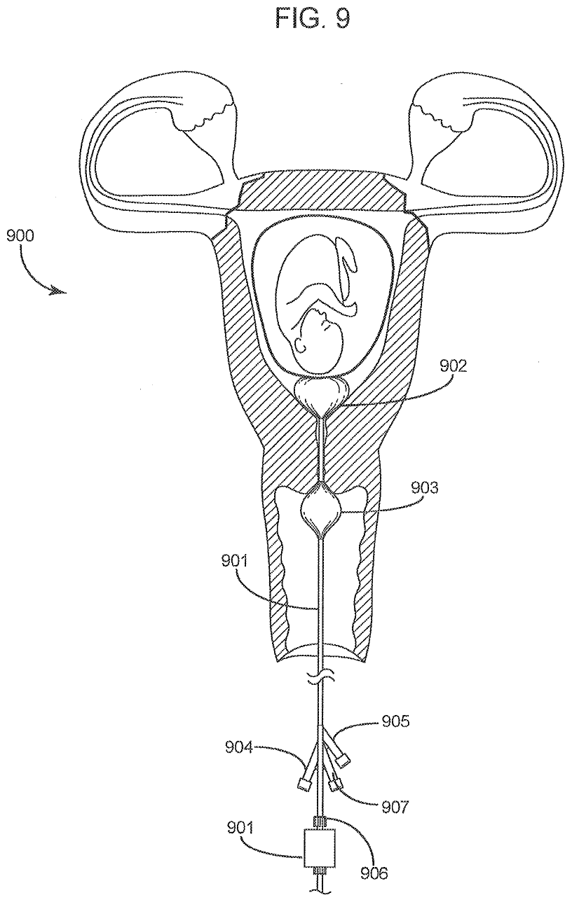

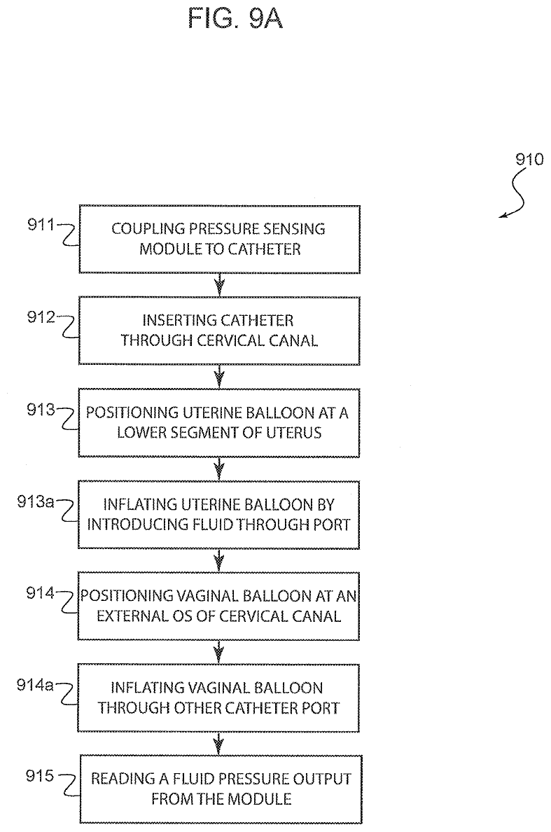

Turning now to the next figure, FIG. 6A a similar exemplary embodiment as that shown in FIG. 5, comprising a catheter 601 that may include: a uterine balloon 610 substantially at the distal end 603 and a conduit/tube (not shown) inside the catheter 601, spanning from the proximal end 602 to the distal end 603 and in fluid communication with the uterine balloon 610, and a vaginal balloon 692, in fluid communication with a vaginal-balloon conduit/tube (not shown) inside the catheter 601, spanning from the proximal end 602 to the distal end 603. The vaginal balloon 692, is spaced apparat and situated a distance closer to the proximate end 602 of the catheter 601 so that when catheter 601 is inserted through the cervix of a patient, balloon 610 may be situated at a lower segment of the uterus and balloon 693 may be situated outside of the uterus and against an exterior portion of the cervix anchoring the balloon at or proximate to the external OS 115 of the patient (see also FIG. 9 and related discussion below).

In exemplary embodiments, system 600 includes a catheter 601 configured for manipulation by an operator, the catheter including: a distal end 603 adapted to be placed at least partially inside a cervix of a patient; a balloon 610 situated substantially at the distal end 603 of the catheter 601 adapted to be placed at least partially inside a uterus of the patient; a first port 606 situated at the proximate end of the catheter 601; a second port 607 situated at the proximate end 602 of the catheter 601; and a conduit fluidly communicating the first port 606 and the second port 607 to an interior of the balloon 610, wherein the balloon is inflated by introducing a fluid through the first port 606; and a pressure sensing module 620 coupled to the second port, the pressure sensing module 620 including an enclosure having a first chamber in fluid communication with the conduit, the first chamber including a pressure-sensing membrane in communication with a sensor (see for example FIG. 7, or FIGS. 8A-8C), wherein the pressure sensing module is configured to detect a pressure applied to the uterine balloon 610 of the catheter 601. In exemplary embodiments of the present disclosure, the first port 606 of the catheter may include a valve configured to prevent a fluid injected through port 606 and into the conduit that fills the balloon 610 from returning back out of port 606. Similarly, a first chamber of pressure sensing module 620 may also include a valve, such as a check-valve or otherwise scalable valve 621a, which allows any air inside the chamber to exit the pressure sensing module as the fluid is received. Once adequately filled, a user may seal the valve 621a in order to maintain the fluid within.

As seen in FIG. 6A, the location of the vaginal balloon 692 on the catheter 610 is closer to catheter's proximal end 603 than the location of the uterine balloon 610 is. The vaginal balloon 692 is adapted for positioning within the vagina 112, at a distal portion thereof, for pressing against the external cervical OS 115. The distal portion of the two-balloon catheter of system 600 in such embodiment, from catheter 601's distal end 602, may be inserted into a woman's reproductive system 100, prior to inflation of the balloons.

In an exemplary two-balloon inflatable system 600, the vaginal balloon 692 is at least partly operative to anchor the uterine balloon 692 in place. That is, vaginal balloon 692 will help stabilize uterine balloon 610 so that uterine balloon 610 may rest securely against an unruptured amniotic sac of the patient. In this way, more accurate readings may be obtained. More specifically, when uterine balloon is filled and placed against an unruptured amniotic sac, a pressure change within the unruptured amniotic sac may be detected via the pressure sensing module since any change in pressure within the unruptured amniotic sac will be transferred to the uterine balloon, which is in fluid communication with a pressure sensing membrane of the pressure sensing module 620 (see also FIG. 7). Moreover, as with previously discussed exemplary embodiments, pressure sensing module 620 may be coupled to an external component such as a sensing device or monitor via communication means 611.

In various embodiments, the inflatable system 600 may also have openings, such as opening 688, which is in fluid communication with a conduit/tube (not shown) inside the catheter 601, spanning from the proximal end 602 to the distal end 603. Opening 688 is situated on the catheter 610 such that, for example, upon placement in a woman's reproductive system the opening 688 is positioned within the cervical canal 114 and is used to insert pharmaceutical substances into the cervical canal 114. Similarly, catheter 601 may include a tip 608 that is blunt so as to not rupture the amniotic sac or may be alternatively configured to rupture the amniotic sac. The blunt tip 608 may include an opening 609 so that an instrument, such as a temperature transducer or an imaging transducer, may be provided access to the lower segment of the uterus and amniotic sac of a patient via opening 609.

In accordance with the present disclosure, the inflation of the balloons of the inflatable system 600 may be performed by a doctor or a midwife (not shown) directly after its insertion. Alternatively, the inflation of the balloons of the inflatable system 600 may be performed by the patient via a hand pump, in a gradual manner, for example over several hours.

Without limiting the scope of the present disclosure, a pump may include any kind of manual or automatic gas or fluid pump suitable for inflating the one or more balloons of catheter 610 of system 600.

As a person of ordinary skill in the art will appreciate, several conduits within catheter 601 will facilitate the inflation of the vaginal and uterine balloons, as well as the introduction of devices such as a stylet and or instruments that may be desirably introduced through the catheter. For example, and without limiting the scope of the present disclosure, catheter 601 may include (in addition to the ports mentioned above) several ports 693, 694, and 695. In order to inflate balloon 692, a port 693 may be employed. Furthermore, in order to guide catheter 601 into the desired position, another port 694 may be implemented for inserting a stylet (not shown here but see for example FIGS. 6B-6E). Similarly, an additional port 695 may be used in embodiments in which opening 688 is included in order to provide a means for supplying a pharmaceutical or therapeutic agent via said port 695 through opening 688.

Turning now to the next set of figures, FIGS. 6B-6D illustrate an exemplary stylet that may be implemented with some exemplary embodiments of a catheter in accordance with the present disclosure, which includes a tubular support for facilitating the introduction of other instruments through the catheter. More specifically, these figures show stylet 650, which includes a handle 651 and a substantially hollow tubular support 652 coupled to stylet 650 via at least one detachable arm 653. The hollow tubular support 652 may be detached or broken off from stylet 650, typically by pressure applied by the operator. It is supposed that braking away the stylet leaves the tubular support inside a conduit of the catheter in order to provide support so that opening 609 may be kept accessible to other devices. As mentioned above, other devices may include a temperature transducer or an imaging transducer, which may be provided access to the lower segment of the uterus and amniotic sac of a patient via opening 609. As illustrated in this set of figures, FIG. 6B shows stylet 650 outside of a conduit of catheter 601; FIG. 6C shows stylet 650 inside the conduit and facilitating a positioning of the tubular support 652 within the conduit; and FIG. 6D shows how once within a desired region (for example a region 610a that may support uterine balloon 610), arms 653 may be broken or snapped off from the tubular support in order to remove stylet 650 and leave the tubular support 652 inside the conduit of catheter 601.

Turning now to the next figure, FIG. 7 illustrates a block diagram of an exemplary system employing a pressure sensing module in accordance with some embodiments of the present disclosure. More specifically, FIG. 7 depicts system 700 in accordance with some exemplary embodiments of the present disclosure, which is configured to assist an operator in evaluating an intrauterine pressure during labor before rupturing the amniotic membrane. In some exemplary embodiments, system 700 comprises a catheter 701 having at least one balloon-a uterine balloon 702—in fluid communication with a fluid chamber 703 of a pressure-sensing module 720. The fluid chamber 703 exemplarily includes an impermeable pressure-sensing membrane 704 situated within an enclosure 705 that houses fluid chamber 703. A port 706 of the pressure sensing module 720 communicates a conduit 701c of catheter 701 that fluidly connects balloon 702 to fluid chamber 703. A sensing circuitry 707 is coupled to pressure-sensing membrane 704 and configured to detect a change in pressure applied to balloon 702.

In exemplary embodiments, sensing circuitry 707 typically includes a sensor module 708, a microcontroller 709 having a memory 710 that includes a set of executable instructions for reading sensing data from the sensor module 708, a communications module 711 for transmitting the sensing data to a remote device having a transceiver 712 or directly to a monitor via monitor interface 713, and a power module 714 to supply power to the components of sensing circuitry 707.

In some exemplary embodiments, sensor module 708 may comprise one or more sensors adapted to generate sensing signals concerning a pressure applied to the uterine balloon 702 via sensing a change in pressure applied to pressure-sensing membrane 704. The sensors may include optical sensors, strain gauges, capacitive sensors, Hall Effect sensors, and the like, and may measure stress and/or strain and/or deflection of the pressure-sensing membrane 704.

As may be appreciated from the block diagram, because uterine balloon 702 of catheter 701 is in fluid communication with the pressure-sensing membrane 704, a pressure or force Fp applied to uterine balloon 702 will be transferred to pressure-sensing membrane 704. Accordingly, pulsations of the amniotic sac will be sensed by the sensing circuitry of the pressure sensing module. In this manner, changes in the intrauterine pressure of a patient in labor may be monitored before rupturing the amniotic sac. As briefly mentioned above, an advantage of monitoring intrauterine pressure without rupturing the amniotic sac is a decrease in the risk of intrauterine infections for the mother and fetus during labor that is typically associated with prematurely rupturing the amniotic sac. Moreover, this allows for improved recording of the number of contractions and contraction patters. Further, use of system 700 may further cause the cervix to be diluted and ripen.

In some exemplary embodiments of the present disclosure, system 700 may include: a catheter 701 configured for manipulation by an operator, the catheter 701 including: a distal end adapted to be placed at least partially inside a cervix of a patient; a uterine balloon 702 situated substantially at the distal end of the catheter 701 adapted to be placed at least partially inside a lower segment of a uterus of the patient; a port 701a situated at a proximate end of the catheter 701; a port 701b situated also at the proximate end of the catheter; and a conduit 701c fluidly communicating port 701a and port 701b to an interior of the uterine balloon 702, wherein the uterine balloon 702 is inflated by introducing a fluid through the port 701a; and a pressure sensing module 720 coupled to port 701b, the pressure sensing module including an enclosure 705 having a fluid chamber 703 in fluid communication with the uterine balloon 702 via the conduit 701c, a pressure-sensing membrane 704 coupled to the fluid chamber 703, and optionally a sensing circuitry 707 coupled to the pressure-sensing membrane 704 configured to detect a pressure applied to the uterine balloon of the catheter. In some exemplary embodiments, the sensing circuitry is external to pressure sensing module 720 (so that the sensing circuitry is housed separately). In some exemplary embodiments, the sensing circuitry is integral with the pressure-sensing module 720 so that the sensing circuitry is housed together with the other components of pressure-sensing module 720, for example wherein enclosure 705 of pressure-sensing module 720 includes a second chamber that houses sensing circuitry 707.

In some exemplary embodiments of the present disclosure, a pressure sensing module 720 may include: an enclosure 705 configured to couple to port 701b of a catheter 701, wherein the catheter 701 includes: a distal end adapted to be placed at least partially inside a cervix of a patient; a uterine balloon 702 situated substantially at the distal end of the catheter adapted to be placed at least partially inside a uterus of the patient; a port 701a and a port 701b situated at the proximate end of the catheter 701; and a conduit 701c fluidly communicating ports 701a and 701b to an interior of the uterine balloon 702, wherein the uterine balloon 702 is inflated by introducing a fluid through the port 701a. The enclosure 705 houses fluid chamber 703 within the enclosure 705 and includes chamber port 706 adapted to couple with port 701b for achieving fluid communication between fluid chamber 703 and the uterine balloon 702 via the conduit 701c. In exemplary embodiments of the present disclosure, the port 701a of catheter 701 may include a valve (not shown) configured to prevent a fluid injected through port 701a and into the conduit 701c that fills the balloon 702 from returning back out of port 701a. Similarly, fluid chamber 703 of enclosure 705 may also include a valve, such as a check-valve or otherwise a scalable valve 705a, which allows any air inside fluid chamber 703 to exit as the fluid is received therein. Once adequately filled, a user may seal the valve 705a in order to maintain the fluid within fluid chamber 703, conduit 701c, and balloon 702.

Moreover, pressure-sensing module 720 includes a pressure-sensing membrane 704 coupled to the fluid chamber 703, and a sensing circuitry 707 coupled to or in communication with the pressure-sensing membrane 703, said sensing circuitry 707 configured to detect a pressure applied to the uterine balloon 702 of the catheter 701.

Now turning to the next set of figures, FIG. 8A illustrates an exemplary pressure sensing module 820 in accordance with some exemplary embodiments of the present disclosure. In the shown embodiment, the pressure sensing module 820 includes an enclosure 821 having a valve 821a, first chamber 822 in fluid communication with the conduit 829, a second chamber 823 in fluid communication with a sensor 826, and an pressure-sensing membrane 824 separating the first chamber 822 from the second chamber 823, wherein the pressure sensing module 820 is configured to detect a pressure applied to the balloon 810 of the catheter 801. In some exemplary embodiments, such as the one depicted in FIG. SA, the second chamber 822 of the pressure sensing module 820 houses a second fluid that is distinct from the fluid introduced through the first port 806 into the first chamber 822. In some exemplary embodiments, the fluid in the second chamber 823 is a gas or a liquid. In some exemplary embodiments, a gas in the second chamber may be simply air. In some exemplary embodiments, the fluid introduced through the first port 806 into the first chamber 822 is water or a nontoxic liquid.

Now turning to the next figure, FIG. 8B illustrates pressure sensing module 820 in accordance with some exemplary embodiments of the present disclosure. In embodiments such as the one depicted in this figure, a pressure sensing module 820 for monitoring and measuring a change in intrauterine pressure during labor, without rupturing the amniotic sac, may include an enclosure 821 configured to couple to a first port 807 of a catheter 801, the catheter 801 including: a distal end 803 adapted to be placed at least partially inside a cervix of a patient; a balloon 810 situated substantially at the distal end 803 of the catheter adapted to be placed at least partially inside a uterus of the patient; a second port 806 situated at the proximate end 802 of the catheter 801; and a conduit fluidly communicating the second port 806 and the first port 807 to an interior of the balloon 801, wherein the balloon 801 is inflated by introducing a fluid through the second port 806; a first chamber 822 housed within the enclosure 821 and in fluid communication with the conduit; a second chamber 823 housed within the enclosure 821 and in fluid communication with a sensor 826; and an pressure-sensing membrane 824 separating the first chamber 822 from the second chamber 823, wherein the sensor 826 is configured to detect a pressure applied to the balloon 810 of the catheter 801. In the shown embodiments, air that may be occupying chamber 822 prior to a liquid being introduced, may be ejected out of chamber 822 via a valve 821a.

In some exemplary embodiments, the pressure sensing module 820 may include a second chamber 823 that houses a second fluid that is distinct from the fluid introduced through the second port 806 into the first chamber 822. In some embodiments, the fluid in the second chamber 823 is a gas or a liquid. In some exemplary embodiments, the gas in the second chamber 823 is air.

In some exemplary embodiments, the enclosure 821 of the pressure sensing module 820 includes a third chamber 825 for housing the sensor 826. In some exemplary embodiments, the enclosure 821 is removably coupled to the sensor 826, wherein the sensor 826 is situated external to the enclosure 821.

Now turning to the next figure, FIG. 8C illustrates pressure sensing module 820 in accordance with yet another exemplary embodiment of the present disclosure. In embodiments such as the one depicted in this figure, a pressure sensing module 820 for monitoring and measuring a change in intrauterine pressure during labor, without rupturing the amniotic sac, may include a second chamber 823 configured to house the sensing circuitry 826, which is coupled to the pressure-sensing membrane 824 without any interface fluid in-between. In some exemplary embodiments, sensing circuitry 826 may employ transmitters or transceivers and the like to communicate with a remote monitor or monitoring device 828.

In some exemplary embodiments, sensing circuitry 826 may comprise one or more sensors adapted to generate sensing signals concerning a pressure applied to the pressure-sensing membrane 824. The sensors may include optical sensors, strain gauges, capacitive sensors, Hall Effect sensors, and the like, and may measure stress and/or strain and/or deflection of the pressure-sensing membrane 824.

Monitoring device 828 may facilitate presenting sensing data including but not limited to changes in the intrauterine pressure of a patient in labor, which may be monitored before rupturing the amniotic sac. As briefly mentioned above, an advantage of monitoring intrauterine pressure without rupturing the amniotic sac is a decrease in the ration of intrauterine infections for the mother and fetus during labor. Moreover, a monitoring device 828 may facilitate recording of the number of contractions and visually appreciating contraction patters that will be useful to the operator during the patient's labor.

Turning now to the next figure, FIG. 9 illustrates an exemplary multi-balloon catheter system in accordance with the present disclosure, shown inside a woman's reproductive system. More specifically, FIG. 9 depicts system 900, comprising: a catheter 901 including a uterine balloon 902, as well as a vaginal balloon 903 situated below or closer to the proximate end of the catheter than the uterine balloon and adapted to secure the uterine balloon inside the uterus; more specifically vaginal balloon 903 secures catheter 901 so that uterine balloon 902 may be more stable in its position in the lower section of the uterus. Moreover, catheter 901 includes several ports. In the embodiment shown in FIG. 9, catheter 901 includes ports 904, 905, 906, and 907.

Port 904 may, for example and in no way limiting the scope of the present disclosure, fluidly communicate with a conduit within catheter 901 that may be used by an operator for inserting a stylet that guides the catheter into cervix of the patient. Port 905 may, for example and in no way limiting the scope of the present disclosure, fluidly communicate with a conduit that fluidly connects port 905 to port 906 via an interior of the uterine balloon 902, in a manner such that injecting a fluid into port 906 may inflate balloon 902 as well as reach a chamber within pressure-sensing module 901 fluidly coupled to port 906. Port 907 may, for example and in no way limiting the scope of the present disclosure, fluidly communicate with vaginal balloon 903 so that injecting a fluid via port 907 will inflate vaginal balloon 903.

In some exemplary embodiments, system 900 comprises: a catheter 901 configured for manipulation by an operator, the catheter including: a distal end adapted to be placed at least partially inside a cervix of a patient; a uterine balloon 902 situated substantially at the distal end of the catheter 901 adapted to be placed at least partially inside a uterus of the patient; a vaginal balloon 903 situated closer to the proximate end of the catheter 901 than the uterine balloon 902 and adapted to secure the uterine balloon 902 inside the uterus; a first port 905, a second port 906, a third port 907, and a fourth port 904 situated at the proximate end of the catheter, wherein the third port 907 is in fluid communication with the vaginal balloon 903; and a conduit fluidly communicating the first port 905 and the second port 906 to an interior of the uterine balloon 902, wherein the uterine balloon 902 is inflated by introducing a fluid through the first port 905; and a pressure sensing module coupled to the second port 906, the pressure-sensing module including an enclosure having a chamber in fluid communication with the uterine balloon 902 via the conduit, a pressure-sensing membrane coupled to the chamber, and a sensing circuitry coupled to the pressure-sensing membrane configured to detect a pressure applied to the uterine balloon 902 of the catheter 901.

A method in accordance with practice of the present disclosure, may include a method 910 for monitoring and measuring a change in intrauterine pressure during labor, without rupturing the amniotic sac. With reference to FIG. 9 and FIG. 9A, by way of example and in no way limiting the scope of the present disclosure, such method 910 may include the following steps, disclosed in one particular sequence although other sequences may be practiced without limiting from the scope of the present disclosure.

In step 911, a pressure-sensing module may be coupled to a catheter as described above.

In step 912, the catheter may be inserted into a vagina and through a cervical canal of a female patient, wherein a distal end of the catheter is placed inside the cervical canal, and a proximal end of the catheter is kept outside the cervical canal, the catheter including: a uterine balloon in fluid communication with the proximal end and attached at or near the distal end of the catheter, a first port situated at the proximate end of the catheter, a second port situated at the proximate end of the catheter, a conduit fluidly communicating the first port and the second port to an interior of the uterine balloon, and a vaginal balloon situated closer to the proximate end of the catheter than the uterine balloon and adapted to secure the uterine balloon inside the uterus.

In step 913, the uterine balloon may be positioned at a lower segment of a uterus of the female patient. In exemplary embodiments, subsequent to positioning the uttering balloon at the lower segment of a uterus of the female patient, at step 913a, the uterine balloon may be inflated from the proximal end and through the catheter by introducing a fluid through the first port of the catheter so that the fluid inflates the balloon and enters a first chamber of the pressure-sensing module.

In step 914, the vaginal balloon may be positioned at an external OS of the cervical canal of the female patient, prior to inflating the vaginal balloon. Subsequently, at step 914a, the vaginal balloon may be inflated to anchor the uterine balloon inside the lower segment of the uterus so that the uterine balloon rests against an amniotic sac of the female patient.

In step 915, once the vaginal balloon is secured and the uterine balloon is anchored or secured in place against the amniotic sac of the female patient—as discussed above—a pressure output may be read from the pressure-sensing module, which detects a change in intrauterine pressure or a change in pressure inside the unruptured amniotic sac, without having to rupture the amniotic sac.

In some exemplary embodiments, during or after the procedure or after taking several readings, it may be desirable to take additional measurements with instruments or devices that may be introduced through the catheter. For example, and in no way limiting the scope of the present disclosure, temperature transducers or imaging transducers may be inserted through an opening at the distal end of the catheter.

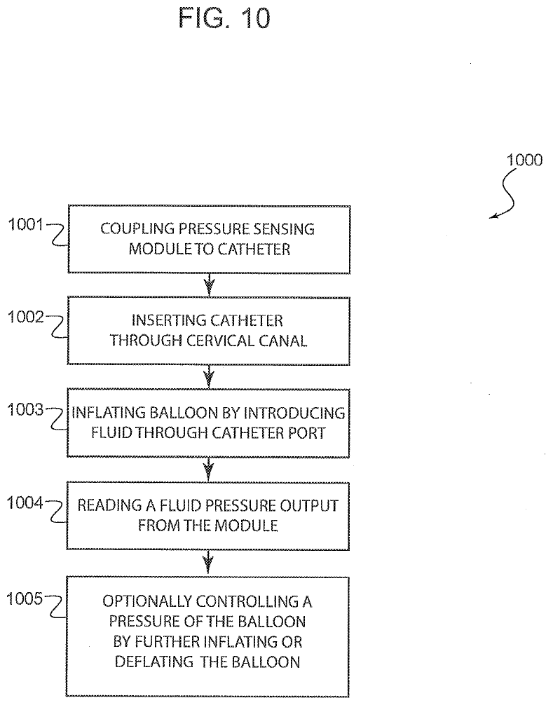

Turning now to the last figure, FIG. 10 depicts another flow chart of a method in accordance with practice of the present disclosure. More specifically, FIG. 10 depicts method 1000, for monitoring and measuring a change in intrauterine pressure during labor, without rupturing the amniotic sac. Although method 1000 is exemplarily shown with a series of steps in one particular sequence, method 1000 may include fewer or more steps in alternative sequences without deviating from the scope of the present disclosure.

In exemplary embodiments, a method for monitoring and measuring a change in intrauterine pressure during labor, without rupturing the amniotic sac, may include steps 1001-1005. In exemplary embodiments, step 1001 may include coupling a pressure sensing module to a catheter.

Step 1002 may include inserting the catheter into a vagina and through a cervical canal of a female patient, wherein: a distal end of the catheter is placed inside the cervical canal, and a proximal end of the catheter is kept outside the cervical canal, the catheter including: a balloon in fluid communication with the proximal end and attached at or near the distal end of the catheter, a first port situated at the proximate end of the catheter, a second port situated at the proximate end of the catheter, and a conduit fluidly communicating the first port and the second port to an interior of the balloon. In some exemplary embodiments, the catheter may include a plurality a plurality of balloons and accordingly this step may further include: a first balloon is placed inside the uterus, and a second balloon is placed inside the vagina. In some exemplary embodiments, the catheter may include a plurality a plurality of balloons and accordingly this step may further include: a first balloon is placed inside the uterus, a second balloon is placed inside the vagina, and a third balloon is placed inside the cervical canal.

In exemplary embodiments, step 1003 may include inflating the balloon from the proximal end and through the catheter by introducing a fluid through a first port of the catheter so that the fluid enters inflates the balloon and enters a first chamber of the sensing module.

In exemplary embodiments, step 1004 reading a pressure output from the pressure sensing module.

In exemplary embodiments, step 1005 optionally controlling a pressure of the balloon by further inflating or deflating the balloon. In some exemplary embodiments, this step may comprise manually inflating or deflating the balloon. In some exemplary embodiments, this step may comprise automatically inflating or deflating the balloon, by for example implementation of a pump device.

As mentioned above, the presented embodiments disclose an inflatable system that may use a single uterine balloon, a uterine balloon and a vaginal balloon, or a uterine and a vaginal and a cervical balloon, or any combination thereof. In various embodiments, each of the named balloons (vaginal, uterine, cervical) may itself be a combination of several balloons. In some embodiments the uterine balloon and/or the cervical balloon are inflated by incompressible fluids, such as water or oil, to transfer the intracorporeal pressures to extracorporeal pressure sensors. While fluid is the best medium for transfer of pressure throughout the system, air or other gases may also be used for this purpose. In all applications, nontoxic liquids or gases are preferable.

The balloons, which may have rough external surfaces in order to keep them anchored in place, may be inflated by the operator or by the patient herself. Various sensors and other instruments may be used along with or as a part of the inflatable system to monitor cervical dilation, fetal well-being, and the woman's conditions. The disclosed inflatable system is not limited in its application to the details of construction and the arrangement of the components set forth in this specification or illustrated in the drawings.

In another aspect of the disclosure, there is provided a multi-lumen catheter for the purpose of accelerating the time for dilation during delivery. In addition, the device will provide patient data on both the mother and fetus with or without the presence of the amniotic sac. Patient data will include, time, duration and intensity of uterine contractions; fetal heart rate; and fetal pulse oximetry (SPO2).

The elongate catheter body may be an extrusion and includes multiple lumens to house or conduct signals to or from sensors to measure uterine contractions, sensors to measure fetal heart rate and pulse oxygenation, uterine balloon expansion, vaginal balloon expansion. At least one open lumen is preferably provided as a working channel to enable multiple uses such as stylet use and access to the amniotic sac with various diagnostic or therapeutic tools. The extrusion may be a co-extrusion with two different durometer materials to add stiffness for insertion and radial support under the balloons while maintaining compliance for maneuverability.

For example, one of an inner tubular wall or an outer tubular wall may comprise an embedded support tube having relatively higher crush resistance. The support tube may extend the entire length of the catheter, or may have a length of less than about 10 cm or less than about 5 cm or no more than about 1 cm or 2 cm longer than the balloon that it is intended to support. As has been previously discussed, the support tube can be located under the uterine balloon and/or the vaginal balloon and allow inflation of the balloon while maintaining full patency of the underlying working channel or other collapsible structure. The support tube may be a polymeric tube having a higher durometer than the adjacent catheter body, and may be formed as a coextrucion. Alternatively an embedded polymeric or metal (e.g., stainless steel or Nitinol) spring may be provided to increase crush resistance in response to inflation of the balloon or other compressive forces that may be encountered. The large diameter of the extrusion will also add stiffness limiting the need for a stylet to place the device.

Referring to FIG. 11, there is schematically illustrated a catheter 909 in accordance with the present disclosure. The catheter 909 includes an elongated flexible tubular body 901, extending between a proximal end and a distal end. A uterine balloon 902 is spaced distally apart from a vaginal balloon 903. A manifold 920 at the proximal end of the tubular body 901 is provided with a vaginal balloon port 922 in fluid communication with the vaginal balloon 903 via a vaginal balloon inflation lumen 936. A working channel port 924 is in communication via a working channel 938 which extends throughout the length of the catheter to a distal opening, providing access throughout the length of the tubular body 901 as has been discussed. A uterine balloon port 926 is in fluid communication with the uterine balloon 902 via a uterine balloon lumen 940. A pressure sensor connector 928 is in fluid communication with a pressure sensor, discussed in further detail below. Preferably, the distal end of the tubular body 901 carries a sensor array 930 such as for determining fetal heart rate, SPO2 and additional options discussed below depending upon the desired functionality.

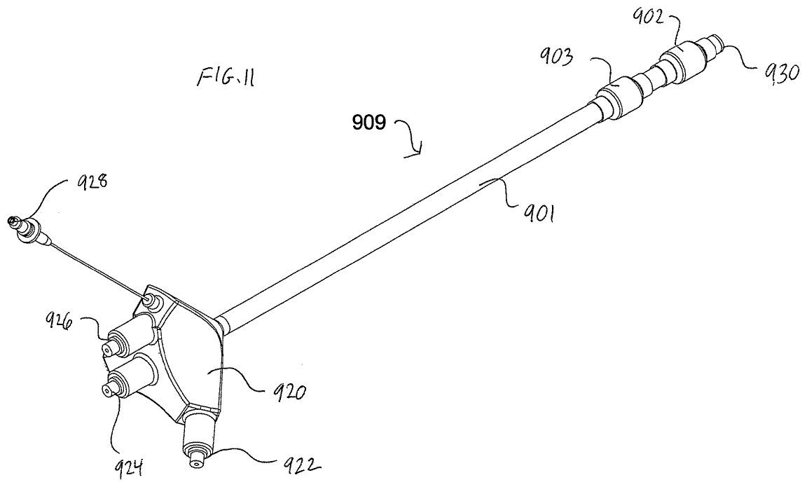

FIG. 12 illustrates a side elevational cross-section through a distal portion of the catheter 909. The tubular body 901 carries a vaginal balloon 903 in communication with the vaginal balloon inflation lumen 936 by way of a vaginal balloon fill port 942. The uterine balloon 902 is in fluid communication with the uterine balloon inflation lumen 940 by way of a uterine balloon fill port 944. A pressure sensing balloon 946 is provided in pressure sensing communication with the uterine balloon 902 and uterine balloon inflation lumen 940. A distal sensor array 930 carrying any of a variety of emitters, detectors or other sensors discussed further below, may be secured to a distal end of the tubular body 901 such as by a distal end cap 934.

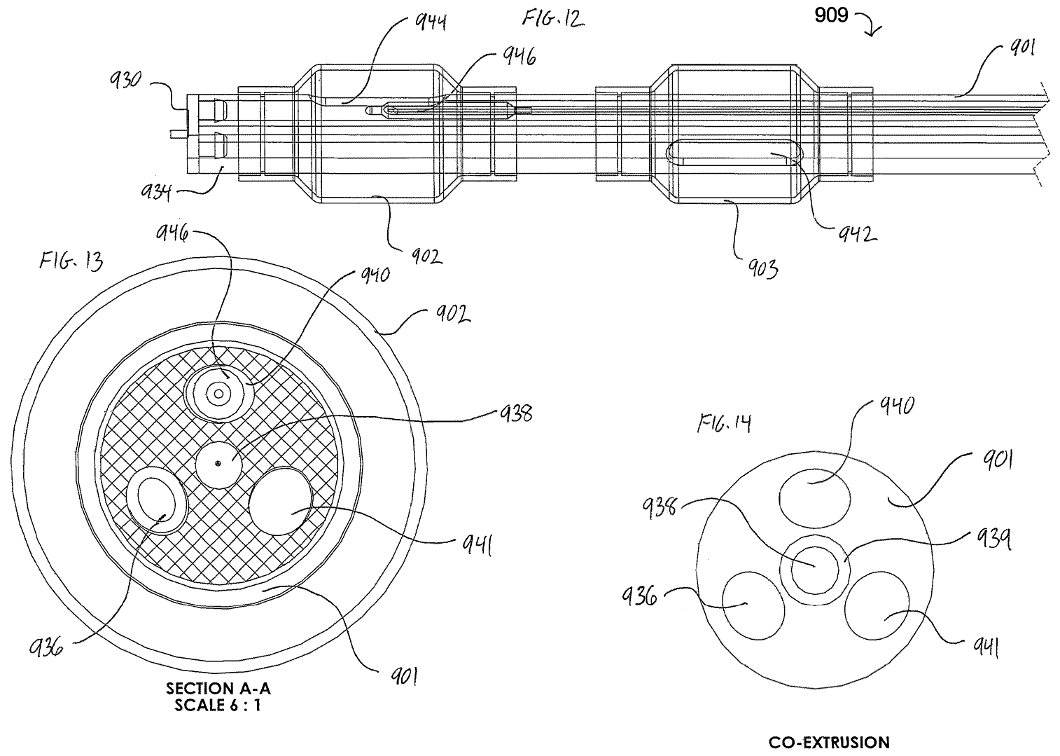

FIG. 13 shows a transverse cross section through a proximal portion of the uterine balloon 902 at a point where it axially overlaps with a pressure sensing membrane such as a sidewall of the pressure sensing balloon 946. Pressure sensing balloon 946 is carried on the distal end of a tubular body 947 which extends proximally throughout the length of the uterine balloon inflation lumen 940. A pressure sensing lumen 949 extends through the tubular body 947 and places the pressure sensing balloon in pressure propagating communication with pressure sensor port 928 at the proximal end of the catheter.

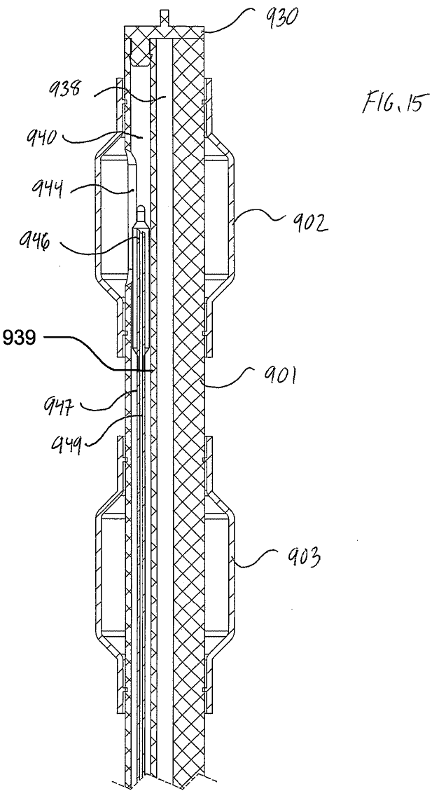

FIG. 14 is a cross section through the multi lumen extrusion of catheter body 901. The extrusion may comprise a soft polymeric material to provide flexibility as has been discussed. As a consequence, inflation of the uterine balloon 902 or vaginal balloon 903 above a threshold pressure may produce sufficient radially inwardly directed pressure to partially or completely collapse the working channel 938. This would interfere with the ability to use the working channel for its intended purpose. As has been discussed, a tubular reinforcement structure such as a spring coil or higher durometer polymer can be embedded in the sidewall to enhance collapse resistance. FIG. 14 illustrates a coextrusion having an inner relatively more rigid, collapse resistant tubular support layer 939 surrounded by the outer, softer extrusion of body 901. The support layer extends at least the length of the uterine balloon and optionally also the vaginal balloon. The coextrusion can extend the entire length of the catheter, and help with column strength.

The distal sensor array 930 may include a distal support such as a PCB and may be provided with one or more typically distally facing sensors which may be mounted at the distal end of the catheter directly in line with the fetus head and configured to measure, for example, fetal heart rate and/or pulse oxygenation.

PPG (photoplethysmography) sensors use a light-based technology to sense the rate of blood flow as controlled by the heart's pumping action. Green LED may be desirable for HR monitoring due to a relatively good signal/noise ratio and better movement sensitivity. Red LED may be desirable for HR monitoring and also SP02. Infra-Red (IR) LED may be also desirable for HR monitoring as well as SP02.

ECG (electrocardiogramaensors may be utilized to measure the bio-potential generated by electrical signals that control the expansion and contraction of heart chambers. Ultra Sound (US), Doppler measurements may be taken to identify blood flow characteristics. Other optional features include a camera (e.g., CMOS or CCD sensor) on the catheter distal end (w/PPG sensor), or a vaginal PPG probe (pre-labor fetal HR measurement device).

The distal end of the catheter may thus be provided with any of a variety of components, depending upon the desired clinical performance. The components may be carried by a support which may be attached to the tubular body 901 by adhesives, slip fit or mechanical interference fit, or as illustrated with an over molded tip 922. The support and components are preferably integrated into the catheter to provide a one time use sterilizable and disposable system.

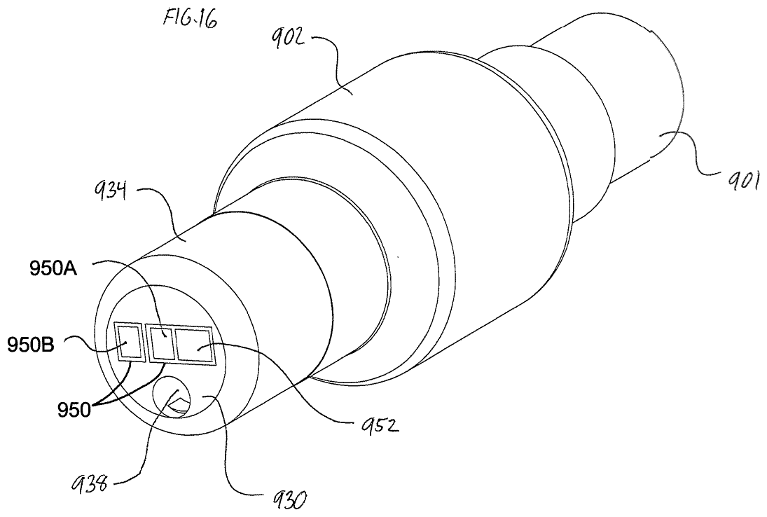

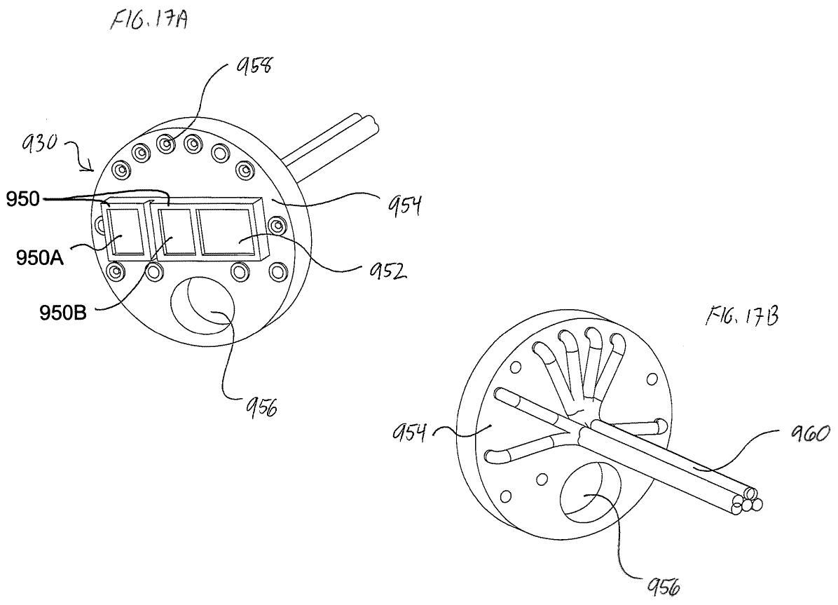

Referring to FIGS. 16-17B, a first component carried by the support may be at least one emitter 950. At a minimum for a PPG catheter the sensing tip would include at least one LED emitter having a narrow or broad wavelength range in the infrared range through the visible optical range of green light or any appropriate subset of the range. The principle of PPG (Photoplethysmography) is that reflection or transmission light changes with blood volume in the microvasculature of tissue. So the PPG signal contains much valuable information about cardiovascular system such as SpO2, heart rate, blood pressure, and respiratory rate. In general, infrared and visible red may be preferred for SPO2 analysis and green may be preferred for heart rate monitoring. Blue light may be preferred for further sensing including sensing the pH of amniotic fluid. In the illustrated embodiment, a first emitter 950A and a second emitter 950B are provided.

A second component carried by the support may be at least one optical sensor such as a photo detector 952. The sensor could be configured to be specific to one wavelength range or could be multiple sensors together that sense a broad range of light depending upon the desired configuration.

It may also be desirable to obtain a pH reading at the distal end of the catheter, which correlates to the pH of fetal blood which allows detection of fetal acidosis during labor. One suitable pH sensor contains a fluorescent dye that experiences a dual excitation maxima. The emitter emits a blue light followed by a violet light to excite the dye at both its excitation wavelengths. The dye then emits light back at a different wavelength. When the sensor is in a solution with a higher pH, more of the deprotonated form (conjugate base) will be present, which leads to increased intensity of the blue light (457 nm) and decreased intensity of violet light (405 nm). When the solution is more acidic, the opposite happens. In accordance with a ratiometric fluorescence measuring technique, the ratio of the intensities at the two excitation wavelengths reveals changes in the pH of the sensor's local environment. Additional details may be found in U.S. Pat. No. 11,071,481 to Dorsey, et al. the disclosure of which is hereby incorporated by reference in its entirety herein.

It may also be desirable to provide at least one ultrasound transducer to enable pelvimetry measurement of the dimensions of the pelvis may be desirable to help determine whether a woman can give birth normally or will require a Caesarean section, e.g., interspinous distance (distance between Ischial spines) and pelvic inlet. An ultrasound transducer may be carried on the support as an integral part of the catheter. Alternatively, the ultrasound transducer may be carried by a separate ultrasound probe which is advanceable through the central lumen only when desired, and perhaps be configured as a sterilizable, reuseable device. Thus This may be accomplished by advancing an ultrasound catheter through the central lumen for positioning the ultrasound transducer at an image capture site for imaging the surrounding bony structure and determining bony pelvic diameter. One suitable imaging catheter is disclosed in U.S. Pat. No. 9,381,027 to Nita et al., entitled Steerable Ultrasound Catheter, issued Jul. 5, 2016, the disclosure of which is hereby incorporated by reference in its entirety herein.

Components intended to be permanently carried by the catheter would be interconnected as necessary on the catheter and wired directly to the handle of the catheter. The handle of the catheter would have a connector that enables the connection to a PCB and a power supply control box. The catheter components would be passive until connected to the control box. The distal PCB would perform the signal conditioning and any necessary integration or digital conversion for exporting the signal to various commercial output devices. This could be performed via a wired connection or a wireless connection.

The components on the distal end of the catheter would be housed in such a way that the optical transmittance and reflectance is optimized for in-vivo use relative to the fetus and the amniotic sac. This could include positioning more than one component in an array or a physical position that ensures good signal acquisition (e.g., distally facing sensors, exposed distally). Programming could be done so that different emitters are pulsed on and off at varying frequencies depending on anatomical differences of amniotic fluid, fetus hair covering, maternal amniotic membrane. The distal surface may be flat and emitting and sensing components are forward looking. Alternatively, the distal tip may be domed in a concave or convex shape and the sensors are aimed on radially inward or outward inclined directions.

An additional sensor may be positioned on the catheter shaft 901 in such a position that it would in direct contact with maternal tissue to provide a second maternal signal. For example, another PPG sensor may be mounted on the shaft such as proximal to the vaginal balloon 903 or in between the vaginal balloon 903 and uterine balloon 902 where it will be in contact with maternal tissue. This provides an independent maternal SPO2 and HR signal within the same device.

Pressure may be measured in the uterine balloon lumen 940 such as with a pressure sensing balloon 946 or other pressure sensor, in pressure propagating contact with the uterine balloon 902 or uterine balloon inflation lumen 940, inside of or outside of the catheter. The on board pressure sensing balloon 946 may be an air filled or liquid (e.g., water filled) balloon.

Referring to FIGS. 12, 13 and 15, the pressure sensing balloon 946 may be located within the catheter shaft in pressure sensing communication with the uterine balloon. In the illustrated implementation, the pressure sensor is positioned within the uterine balloon inflation lumen 940. At least a portion of the pressure sensing balloon 946 may be positioned under the uterine balloon 902 (axial overlap). The pressure sensing balloon 946 is in communication via a pressure sensor lumen with a proximal pressure sensor connector 928.

The entire catheter body 901 is a flexible multi lumen extrusion, such as silicone. If pressure is measured at the proximal end of the catheter without the present distal pressure sensing balloon and shaft, the length and softness of the shaft 901 would dampen the pressure signal and slows response time. So the non-compliant flexible, air filled, pressure sensing balloon is preferably as physically close to the contractions as possible.

The pressure sensing balloon shaft 947 is thus preferably relatively stiff to optimize propagation of pressure waves proximally through the catheter. In one implementation, the shaft 947 comprises relatively stiff nylon as opposed to the flexible silicone of the catheter shaft 901. Preferably the pressure sensing shaft is at least about 5 times or at least about 10 times or at least about 12 times the stiffness of the Silicone shaft or greater. The Silicone shaft may have an outside diameter within the range of about 8 mm to about 12 mm, and in one implementation about 10 mm, while the sensor tube 947 is no more than about 2 mm or about 1 mm in diameter with thin walls. This factored with the difference in material properties alone is probably a 10-100× factor difference for shaft stiffness.

The pressure sensing balloon itself is more of a non compliant (e,g, PET) or low compliance (e.g., nylon; polyurethane) dilatation balloon so significantly stiffer than the relatively compliant cervical balloon.

A separate pressure detector can be attached to the pressure sensor connector 928 to enable detection of pressure and enable display of pressure and pressure changes when the uterine balloon is compressed by the contracting uterus.

In one implementation of the disclosure, the catheter is intended for one time use and then disposal. Certain of the components such as the ultrasound transducer or fetal blood pH sensor or others identified above may be moved to a separate one time use or sterilizable, reusable probe that can be advanced through the working channel as desired, then removed to leave the working channel available for other devices. This can reduce the cost of the catheter and reduce the volume of the on board electrical components.

The sensor data can be used to calculate and measure the time, duration, and intensity of uterine contractions. In addition the pressure sensor can be used as a safety measure during catheter insertion and placement. Pressure spikes during balloon inflation may indicate the balloon is forcing the fetus upwards which may lead to umbilical cord impingement. Pressure sensing during balloon inflation will warn practitioners of any excessive force being placed on the mother and or fetus. In addition the pressure sensor can also be used to optimize the fetal heart rate sensor by setting a pressure target during balloon inflation to ensure proper sensor placement relative to the fetus head.

The open ended working channel 938 serves multiple purposes during the procedure. It allows for the use of a stylet to shape the catheter for easier safer insertion. It will allow practitioner access to the patient's uterus for multiple uses including intra uterine visualization through endoscopy, manual breaking of the amniotic sac, fluid sample collection, and drug delivery.

Additional detail of the distal sensor array 930 may be seen in connection with FIGS. 17A and 17B. The sensor array 930 is preferably carried by a support such as a printed circuit board 954. The printed circuit board 954 may be formed or cut in a circular configuration, to cooperate with the circular catheter shaft.

At least one emitter such as LED 950 is carried by the PCB 954. In the illustrated implementation, a first LED 950 and a second LED 950 along with a photo detector 952 are carried by the PCB. More or fewer sensors of different varieties may be incorporated into the sensor array 930 and carried by the PCB 954, depending upon the intended clinical performance.

The PCB 954 includes at least one through lumen 956 or other cut out to permit the working lumen 938 to open all the way through the distal end of the catheter to permit access to the amniotic sac. A plurality of wire connectors 958 provide junctions for connection of components on the PCB 954 to a plurality of conductors 960 which extends proximally throughout the length of the catheter to appropriate proximal connectors for connection to a control module including a power supply, data processing capabilities and input/output devices such as visual display or keyboard.

The PCB 950 may additionally be provided with at least one processor, for fully or partially processing data from the sensors at the distal end of the catheter, prior to transmitting fully or partially processed data proximally through the catheter via one or more conductors 960.

Basic Steps in one example of the method include:
1) Uterine balloon and distal catheter inserted into the cervix with or without a stylet. Stylet pulled back (if necessary) and assembly inserted until both balloons are in cervix.
2) Distal balloon (uterine) inflated to approximately 20 ml and pulled back against cervix wall.
3) Proximal balloon inflated to approximately 20 ml. Using pressure both balloons inflated at 20 ml increments until optimal pressure is reached.
4) fHR and fSP02 monitoring begins. Uterine activity monitoring begins.
5) Pressure can be used during ripening to determine if more balloon volume should be added.
6) Center lumen can be accessed for visualization, membrane breakage, drug delivery, or sample collection.
7) Once the water breaks, fluid can be removed from the distal balloon (with pressure measurement as a guide) and fluid added to the proximal balloon to maintain fetal HR and fetal SP02 monitoring as well as uterine activity.

A system and method for child-bearing monitoring and assistance has been described. The foregoing description of the various exemplary embodiments of the disclosure has been presented for the purposes of illustration and disclosure. It is not intended to be exhaustive or to limit the disclosure to the precise form disclosed. Many modifications and variations are possible in light of the above teaching without departing from the spirit of the disclosure.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.”

The scope of the present disclosure is not intended to be limited by the specific disclosures of preferred embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A system for monitoring and measuring a change in intrauterine pressure during labor, without rupturing the amniotic sac, comprising:
a catheter configured for manipulation by an operator, the catheter including:
a distal end adapted to be placed at least partially inside a cervix of a patient;
a uterine balloon situated substantially at the distal end of the catheter adapted to be placed at least partially inside a lower segment of a uterus of the patient;
a uterine balloon inflation port situated at the proximal end of the catheter;
a uterine balloon sensing port situated at the proximal end of the catheter; and
a uterine balloon pressure sensor in fluid communication with the inflation port and in pressure sensing communication with the uterine balloon.

2. A system as in claim 1, further comprising a fetal heart rate sensor.

3. A system as in claim 1, further comprising an oxygen saturation sensor.

4. A system as in claim 1, wherein the catheter comprises an extruded catheter shaft.

5. A system as in claim 4, wherein the catheter shaft comprises a coextrusion at least under the uterine balloon.

6. An integrated pressure and fetal heart rate monitoring cervical ripening catheter, comprising:
an elongate, flexible tubular body, having a proximal end, a distal end and at least one working channel extending therethrough;
at least a uterine balloon carried by the tubular body;
an electronic component support mounted on the distal end of the tubular body, and having at least one emitter and at least one sensor.

7. A catheter as in claim 6, further comprising a vaginal balloon on the tubular body, spaced proximally of the uttering balloon.

8. A catheter as in claim 6, comprising a first and second LED, and a photodetector.

9. A catheter as in claim 6, configured to generate data convertible into SpO2.

10. A catheter as in claim 6, configured to generate data convertible into fetal heart rate.

11. A catheter as in claim 6, configured to generate data convertible into pH.

12. A catheter as in claim 6, further comprising at least one ultrasound transducer carried by the electronic component support.

13. A catheter as in claim 6, wherein the working channel extends distally through the support.

14. A catheter as in claim 6, further comprising a maternal PPG sensor on the tubular body, proximal to the uterine balloon.

15. A catheter as in claim 6, further comprising a pressure sensor, for measuring pressure in the uterine balloon.

16. A catheter as in claim 15, wherein the pressure sensor comprises a pressure sensing balloon.

17. A catheter as in claim 16, wherein the pressure sensing balloon is in pressure sensing communication with the uterine balloon.

18. A catheter as in claim 17, wherein the pressure sensing balloon extends between a proximal end and a distal end, and the distal end is distal to a proximal end of the uterine balloon.

19. A catheter as in claim 17, wherein the pressure sensing balloon is carried by a tubular shaft.

20. An integrated pressure and fetal heart rate monitoring cervical ripening catheter, comprising:
a catheter having a proximal end, a distal end, and at least one working channel extending therethrough, the distal end adapted to be placed at least partially inside a cervix of a patient;
a uterine balloon situated substantially at the distal end of the catheter adapted to be placed at least partially inside a lower segment of a uterus of the patient;
a sensing port situated at the proximal end of the catheter in fluid communication with the uterine balloon;
wherein the uterine balloon is configured to be filled by introducing a first fluid through an inflation port;
wherein the at least one working channel is configured to accommodate a sensing probe as the sensing probe is advanced through the distal end of the at least one working channel; and
wherein the sensing port is configured to attach to a pressure-sensing module located extracorporcally from the patient a pressure sensor in fluid communication with the inflation port and in pressure sensing communication with the uterine balloon.
